(19) United States
(12) Patent Application Publication
(10) Pub. No.: US 2019/0201675 A1
(43) Pub. Date: Jul. 4, 2019

(54) ELECTROMOLDED MICRONEEDLES AND FABRICATION METHODS THEREOF

(71) Applicant: National Technology & Engineering Solutions of Sandia, LLC, Albuquerque, NM (US)

(72) Inventors: Philip Rocco Miller, Albuquerque, NM (US); Ronen Polsky, Albuquerque, NM (US); Matthew W. Moorman, Albuquerque, NM (US)

(21) Appl. No.: 16/286,081

(22) Filed: Feb. 26, 2019

Related U.S. Application Data
(62) Division of application No. 15/237,193, filed on Aug. 15, 2016.
(60) Provisional application No. 62/206,062, filed on Aug. 17, 2015.

(51) Int. Cl. A61M 37/00; C25D 1/10; A61M 5/142; A61M 39/22; A61B 5/145; C25D 1/00

(52) U.S. Cl. CPC A61M 37/0015; C25D 1/10; A61M 5/142; A61M 39/22; A61B 5/14503; A61B 2562/12; C25D 1/003; A61M 2037/0023; A61M 2037/003; A61M 2037/0053; A61B 5/14532

(57) ABSTRACT

The present invention relates to microneedles, as well as arrays and methods thereof. In particular, the microneedle is hollow and extends from a flexible substrate. Methods for making such microneedles include depositing electroplating materials within a cavity of a mold and removing an electroplated layer from that mold. In some instances, the mold is formed from an elastomer, which can be removed and reused to produce additional microneedles.

### CROSS-REFERENCE TO RELATED APPLICATIONS

This is a divisional application of U.S. application Ser. No. 15/237,193, filed Aug. 15, 2016, which claims the benefit of U.S. Provisional Application No. 62/206,062, filed Aug. 17, 2015, both of which are incorporated herein by reference.

### STATEMENT OF GOVERNMENT INTEREST

This invention was made with Government support under Contract No. DE-NA0003525 awarded by the United States Department of Energy/National Nuclear Security Administration. The Government has certain rights in the invention.

### FIELD OF THE INVENTION

The present invention relates to microneedles, as well as arrays and methods thereof. In particular, the microneedle is hollow and extends from a flexible substrate. Methods for making such microneedles include depositing electroplating materials within a cavity of a mold and removing an electroplated layer from that mold.

### BACKGROUND OF THE INVENTION

Access to transdermal fluids can provide numerous benefits. For instance, such access can provide an effective means for delivering drugs while minimizing pain. Microneedle technology was initially developed for such drug delivery and can be further applied to other therapeutic and diagnostic uses, such as in wearable sensors.

Fabrication of such microneedles can be challenging. Particular structural features of the microneedle can be optimized for transdermal access or pain minimization, but manufacturing such features on the microscale can be difficult. In addition to performance concerns, fabrication processes should employ biocompatible materials, thereby minimizing inflammatory responses when used on a human patient. Accordingly, there is a need for additional microneedle assemblies and methods of making such assemblies to address these concerns.

### SUMMARY OF THE INVENTION

The present invention relates to microneedles, as well as arrays including such microneedles and methods for making such microneedles and arrays. In one non-limiting instance, the fabrication method was developed for the creation of conformal hollow metal microneedle arrays made using reusable molds. In this method, micromolding and electroplating techniques were used with a process referred to as electromolding. Master structures can be initially fabricated with any useful process, e.g., a two-photon polymerization using laser direct write. Such masters can then be molded with conventional micromolding techniques. Then, molds can be used to create the microneedles by first coating with a seed layer (e.g., a seed layer including Ti and then Au) and then electroplating with another material (e.g., iron, nickel, or alloys thereof) in order to create hollow microneedles. As described herein, an exemplary method was investigated for its creation of hollow microneedles with an off-set bore, for mold replication and reusability, for flexibility of the microneedle substrate, for microneedle fracture strength, and for insertion studies including ex vivo experiments. Additional details follow.

### Definitions

As used herein, the term “about” means +/−10% of any recited value. As used herein, this term modifies any recited value, range of values, or endpoints of one or more ranges.

By “fluidic communication,” as used herein, refers to any duct, channel, tube, pipe, chamber, or pathway through which a substance, such as a liquid, gas, or solid may pass substantially unrestricted when the pathway is open. When the pathway is closed, the substance is substantially restricted from passing through. Typically, limited diffusion of a substance through the material of a plate, base, and/or a substrate, which may or may not occur depending on the compositions of the substance and materials, does not constitute fluidic communication.

By “micro” is meant having at least one dimension that is less than 1 mm. For instance, a microstructure (e.g., any structure described herein) can have a length, width, height, cross-sectional dimension, circumference, radius (e.g., external or internal radius), or diameter that is less than 1 mm.

As used herein, the terms “top,” “bottom,” “upper,” “lower,” “above,” and “below” are used to provide a relative relationship between structures. The use of these terms does not indicate or require that a particular structure must be located at a particular location in the apparatus.

Other features and advantages of the invention will be apparent from the following description and the claims.

### BRIEF DESCRIPTION OF THE DRAWINGS

FIGS. 1A-1B show exemplary methods for forming one or more electromolded microneedles. Provided are schematics of (FIG. 1A) an exemplary method 100 including a mold 110 and (FIG. 1B) another exemplary method 1000 including casting a mold 1100 from a master 1400.

FIGS. 2A-2E show exemplary methods of casting a mold and providing a microneedle. Provided are schematics of (FIG. 2A) a method including casting 201 a mold and cutting 205 to provide an orifice 252 at the distal end of the microneedle; (FIG. 2B) an exemplary master 2400 having an inward ledge 2401; (FIG. 2C) cross-sectional views of an exemplary master along line 2C-2C in FIG. 2B, as well as following steps to provide a microneedle 2500; (FIG. 2D) an exemplary microneedle 2500 formed with an orifice 2501 provided by the inward ledge 2401 feature of the master 2400; and (FIG. 2E) a cut-away view of the exemplary microneedle 2500 showing the internal hollow bore 2504.

FIGS. 3A-3D show other exemplary methods of casting a mold and providing a microneedle. Provided are (FIG. 3A) an exemplary master 340 having an inward ledge 341; (FIG. 3B) cross-sectional views of an exemplary master along line 3B-3B in FIG. 3A, as well as an exemplary resulting mold 310; (FIG. 3C) an exemplary master 3400 having an outward ledge 3401; and (FIG. 3D) cross-sectional views of an exemplary master along line 3D-3D in FIG. 3C, as well as an exemplary resulting mold 3100.

FIGS. 4A-4C show different master designs. Provided are two ledge designs, including (FIG. 4A) an inward ledge and (FIG. 4B) an outward ledge, where the progression is shown beginning from the STL design (left), to the microphotograph of the microneedle master (center), and to the he microphotograph of the electroformed hollow microneedle array (right). Also provided is (FIG. 4C) a microphotograph of an array of microneedles.

FIG. 5 shows a cross-sectional schematic of the electromolding process for hollow microneedle fabrication. The process includes (1) creating of a microneedle (MN) master with two-photon polymerization using laser direct write; (2) micromolding the master with PDMS to create a mold; (3) removing the master from the mold; (4) electron-beam depositing a seed layer to create a conductive coating and a void behind inward facing ledge for the hollow microneedle bore; (5) electroplating upon the seed layer, thereby providing an electroform; and (6) removing the electroform from the mold.

FIGS. 6A-6B show (FIG. 6A) a graph of the resulting bore height within a mold following Ti/Au seed layer deposition with varying ledge sizes and (FIG. 6B) optical images of voids from seed layer within molds. Scale bar is 100 μm.

FIGS. 7A-7B show (FIG. 7A) a graph comparing the designed ledge size to the actual fabricated ledge size and (FIG. 7B) in-mold images of molds made from microneedle masters. Scale bar is 100 μm.

FIGS. 8A-8B show the effect of the mold on tip survival of the microneedle. Provided are (FIG. 8A) an optical image of a hollow microneedle made from a 50 μm ledge PDMS mold with a ratio of 20:1 PDMS precursor:catalyst; (FIG. 8B) an optical image of a hollow microneedle made from a 50 μm ledge PDMS mold with a ratio of 10:1 PDMS precursor:catalyst; and (FIG. 8C) a graph regarding the percentage of microneedle tips that survive the demolding process. Scale bar is 250 μm.

FIG. 9 shows scanning electron and elemental analysis of the bore edge for an electroformed hollow microneedle. Provided are (A) a scanning electron microscopy image of the region; (B) an image with elemental coloring overlay; and (C-F) images of each individual element that was characterized, including (C) silicon, (D) titanium, (E) gold, and (F) nickel.

FIGS. 10A-10B show the effects of repeat molds or reused molds. Provided are (FIG. 10A) optical images from within microneedle molds of both the 40 μm and 50 μm ledge sizes for the first mold, thirtieth mold, and molds that have been reused; and (FIG. 10B) a graph comparing ledge surface area for each ledge size for the first month, thirtieth mold, and a reused mold. Scale bar is 100 μm.

FIG. 11 shows mechanical compression testing of hollow microneedles made via electroforming.

FIGS. 12A-12C show the flexibility of the microneedle array. Provided are (FIG. 12A, FIG. 12C) optical images detailing the flexibility of the electroformed parts by flexing (tensile and compressive flexing) of the microneedle substrate in multiple directions, as well as (FIG. 12B) an optical image of the array with no bending. Microneedle array substrates are 20 mm×20 mm

### DETAILED DESCRIPTION OF THE INVENTION

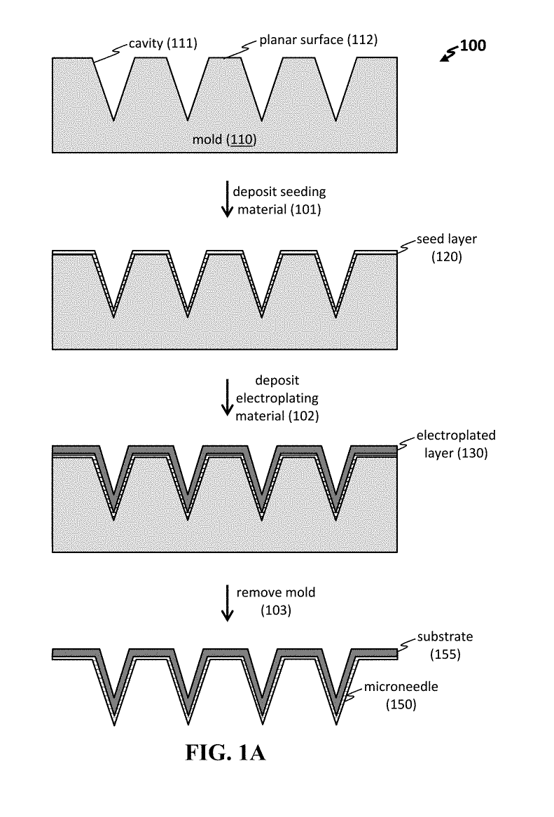

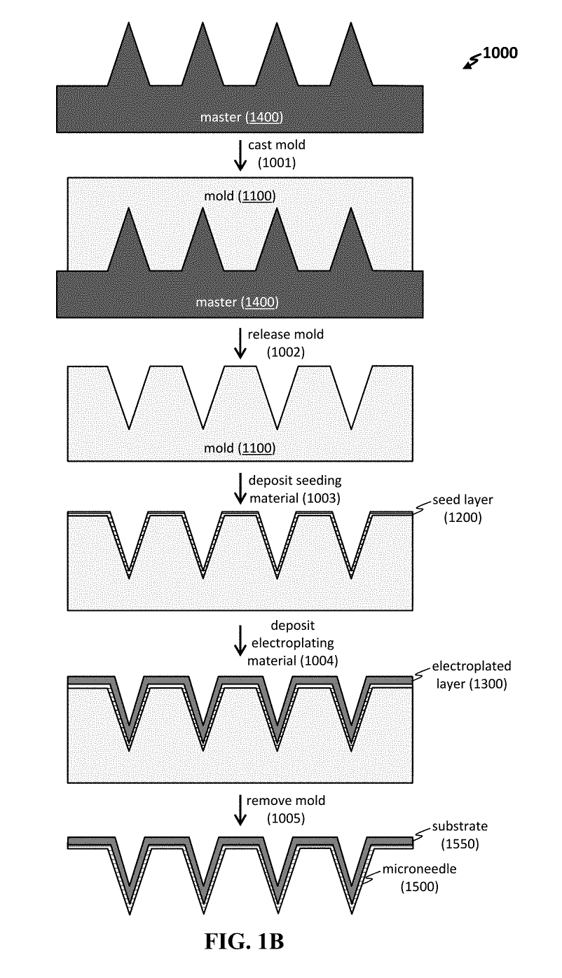

The present invention relates to electromolded microneedles and arrays thereof. Methods of forming such microneedles are also described herein. FIG. 1A provides an array including a plurality of microneedles 150 disposed on (e.g., extending from) a substrate 155. The exemplary method 100 can be used to fabricate the microneedles by employing a mold 110 (e.g., a reusable formed from an elastomer having a sufficiently low Young's modulus to facilitate removal of the electroformed microneedles from the mold, as well as reuse of that mold). The method 100 includes providing a mold 110 having one or more cavities, in which each cavity provides a negative replica of a microneedle. Further steps include depositing 101 one or more seeding materials on a surface, or a portion of a surface, of the one or more cavities 111. To form the substrate, seeding material(s) can be deposited on the substantially planar surface 112 of the mold that is disposed between the cavities 111, thereby forming a seed layer 120 (e.g., a contiguous seed layer that conforms to the surface of the one or more cavities and/or the planar surface). Next, one or more electroplating materials are deposited 102 on the seed layer 120, thereby forming an electroplated layer 130. The electroplated layer 130 includes one or more electromolded microneedles, where each of the electromolded needles includes an internal hollow bore and an orifice disposed at the distal end of the bore. Finally, the mold 110 is removed 103 from the electroplated layer 130 that is bound to the seed layer 120, thereby providing an array of microneedles 150 extending from the substrate 155. The microneedle 150 can include an outer layer and an inner layer, in which the outer layer includes the seed layer (that in turn includes one or more seeding materials) and the inner layer includes the electroplated layer (that in turn includes one or more electroplating materials).

The mold can be formed from any useful master. In this way, the particular geometry and dimension of each microneedle can be optimized with the design of the master, and then this master can be employed to form multiple molds of the master. Then, using the electroplating methodologies described herein, one or more seeding and/or electroplating materials can be deposited within the mold in order to form electromolded microneedles. FIG. 1B provides an exemplary method 1000 including providing the master 1400, casting 1001 one or more polymers in order to form a mold 1100, and releasing 1002 the mold. Then, the mold is employed by depositing 1003 one or more seeding materials in order to form a seed layer 1200 disposed at least within one cavity of the mold 1100. Then, one or more electroplating materials (e.g., any material capable of being electroplated, such a metal) are deposited 1004 upon the seed layer 1200, thereby forming an electroplated layer 1300. Finally, the mold is removed 1005, thereby providing an array of microneedles 1500 extending from a substrate 1550.

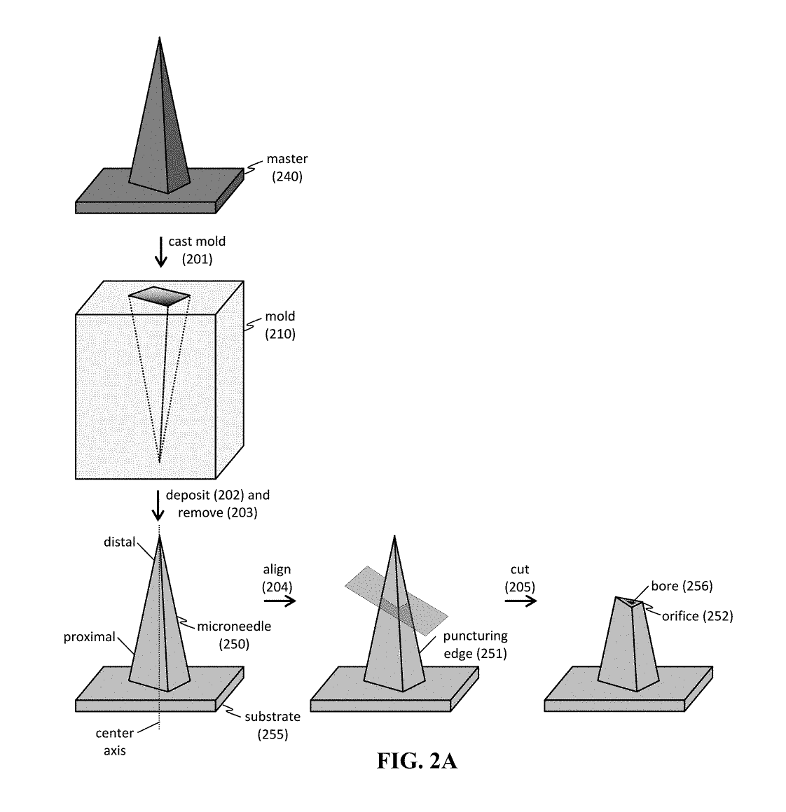

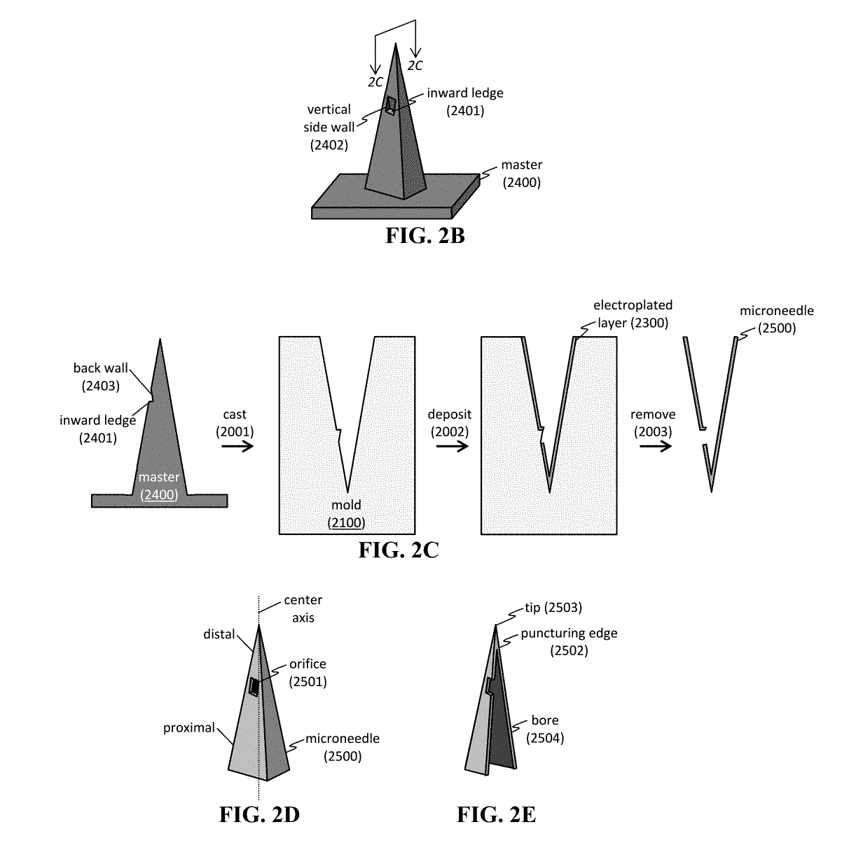

The electroformed microneedles can be hollow, such as by controlling the electrodeposition conditions to ensure that the entire cavity of the mold is not filled and that an empty void is present. In this way, an empty void forms the internal bore of the microneedle once the electroplated layer is removed from the mold. The internal bore of the microneedle can be used to delivery agents to the subject and/or to collect fluids from the subject. To facilitate such delivery and collection, each microneedle can include an orifice located at the distal end of the bore. The orifice can be instilled in any useful manner. In one instance, the orifice is formed by cutting a tip of the electroformed hollow microneedle. FIG. 2A provides an exemplary method, in which the master 240 is provided as a solid structure having any useful configuration (e.g., that is designed for transdermal access). Then, the master 240 is employed to cast 201 a mold 210. One or more seeding materials and/or electroplating materials are deposited 202 within and/or upon the mold, and then the mold 210 is removed 203 to provide a hollow microneedle 250 disposed on the substrate 255. Next, the microneedle is aligned 204 to provide a particular cut angle as compared to the puncturing edge 251. Finally, the aligned microneedle is cut 205 to provide an orifice 252 disposed at the distal end of the hollow bore 256 of the microneedle.

Each microneedle can be characterized by a center axis extending from the distal end to the proximal end of the microneedle. Generally, the tip or puncturing edge of the microneedle is disposed at or in proximity to the distal end, and the substrate is disposed at the proximal end of the microneedle. Each microneedle can be configured to provide fluidic communication between the internal bore and another chamber located in proximity to the substrate, such as by including a port disposed within the substrate and in fluidic communication with the internal bore. One port can be associated with each microneedle, thereby providing individually addressable microneedles.

The master and the resultant mold can be configured to have any useful features. For example, one such feature includes a sharp enough puncturing edge (or tip) that is faithfully replicated by the mold and the resulting microneedle. In another example, the feature includes ledges, protrusion, or openings configured to create an orifice in the microneedle. The orifice can be located in any useful position (e.g., in proximity to the distal end of the microneedle and optionally off-set from the center axis of the microneedle). Many metal deposition processes are directional, meaning that seeding and/or electroplating material(s) are not deposited in a vertical manner but can provide non-vertical side walls. Thus, in some instances, the cavity is not an exact negative replica of the final microneedle. Rather, the cavity includes one or more features (e.g., inward and/or outward ledges) that compensate for directional deposition such that, once depositing the seeding and/or electroplating materials, an orifice is formed.

FIG. 2B provides an exemplary feature on a master 2400, in which the feature is a ledge that provides an orifice for the electroformed microneedle. The inward ledge 2401 is positioned on a face of the master 2400, in which the ledge 2401 is substantially parallel to the plane of the substrate. The feature also includes a vertical side wall 2402, which is substantially orthogonal to a plane of the ledge 2401. As seen in the cross-sectional view in FIG. 2C, the master 2400 also includes a back wall 2403 that connects a surface of the microneedle to the inner edge of the ledge 2401. Next, the method includes casting 2001 an elastomer to provide a mold 2100, which provides a negative replica of the master 2400 and the ledge 2401. As can be seen, the negative replica of the mold 2100 includes a ledge that accumulates one or more seeding materials and/or electroplating materials during the deposition process 2002. In this design, due to accumulation of material on the ledge, such material is not deposited on the replicated back wall, thereby forming an orifice in the microneedle 2500 once the seed layer and electroplated layer 2300 are removed 2003 from the mold.

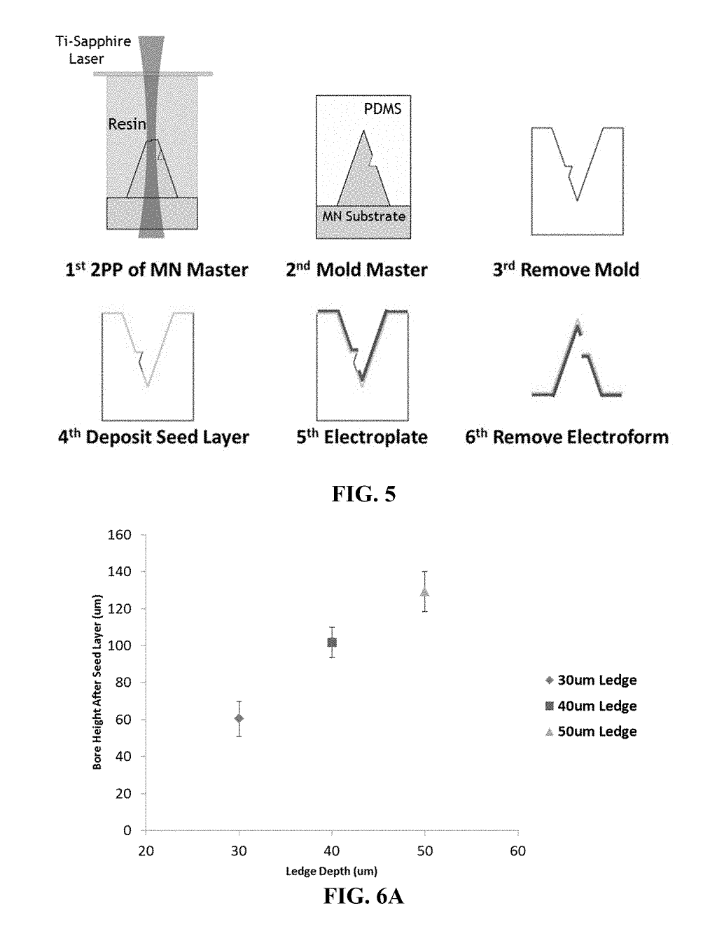

FIG. 2D-2E provides an exemplary microneedle 2500 having an orifice 2501 created a mold having a negative replica of the inward ledge 2401. The orifice 2501 is not located at the tip 2502 of the microneedle but positioned off-set from the center axis and located on a face of the microneedle. To facilitate fluid access, the orifice 2501 is located in proximity to the distal end of the microneedle 2500, as well as in proximity to the puncturing edge 2502. In addition, the orifice 2501 is in fluidic communication with the bore 2504. FIG. 5 provides another exemplary electroforming method.

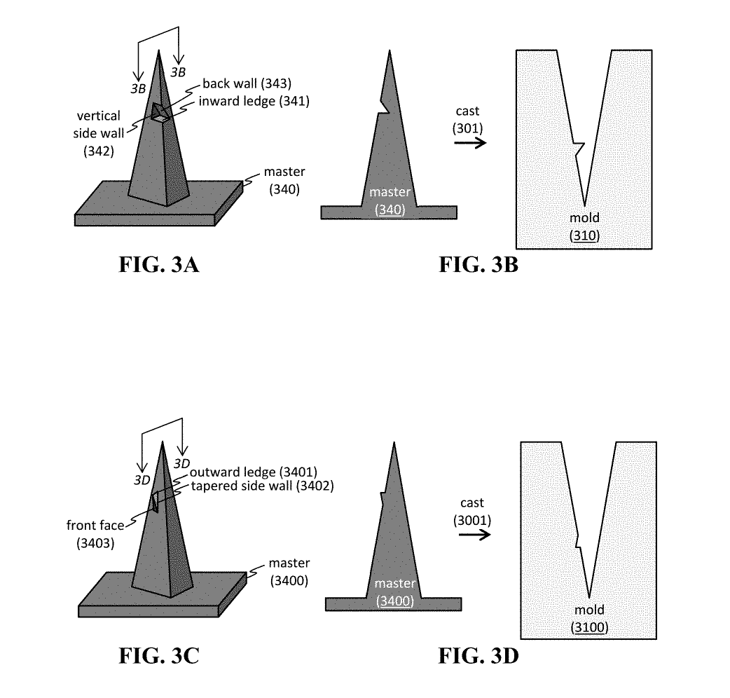

Other master and mold designs can be employed. FIG. 3A provides a master 340 having an inward ledge 241 and a back wall 343 that intersects with a puncturing edge of the microneedle design. The vertical side wall 342 is positioned to be generally parallel to the center axis of the microneedle design. FIG. 3B provides an exemplary mold 310 formed by casting 301 the master 340 with an elastomer. The feature can also include an outward ledge. FIG. 3C provides another exemplary master 3400 having an outward ledge 3401 disposed on a face of the microneedle design. The tapered side walls 3402 connect the ledge 3401 to the face of the microneedle, and a front face 3403 of the feature also connects the ledge 3401 to the face of the microneedle. FIG. 3D provides an exemplary mold 3100 formed by casting 3001 the master 3400 with an elastomer. As can be seen, the mold 3100 includes a negative replica of the ledge 3401 (configured to accumulate seeding and/or electrodepositing materials) and the front face 3403 (configured to minimize deposition, thereby providing an orifice once the electroplated layer is removed from the mold).

Exemplary metal deposition processes include physical vapor deposition (e.g., electron beam physical vapor deposition, cathodic arc deposition, pulsed laser deposition, evaporative deposition, or sputter deposition), chemical vapor deposition (CVD) (e.g., aerosol assisted CVD, microwave plasma-assisted CVD, plasma enhanced CVD, atomic layer CVD, combustion CVD, metalorganic CVD, vapor-phase epitaxy, and hybrid physical-chemical vapor deposition), or thin film deposition (e.g., electroplating, chemical bath deposition, plasma enhanced CVD, atomic layer deposition, electron beam evaporation, molecular beam epitaxy, sputtering, cathodic arc deposition, pulsed laser deposition, etc.). Exemplary metals (e.g., seeding and/or electroplating materials include titanium (Ti), aluminum (Al), nickel, iron (Fe), cobalt (Co), gold (Au), silver (Ag), copper (Cu), platinum (Pt), palladium (Pd), nickel (Ni), chromium (Cr), tungsten (W), molybdenum (Mo), tantalum (Ta), niobium (Nb), as well as alloys thereof (e.g., stainless steel, CoCr, TiAlC, TiAl, TiNi, Au, TiMo, CoCrMo, AuAgCu, or AuPtPd alloys).

### Masters and Molds

The masters and molds can be formed from any useful process. In one instance, the master can be formed using a process capable of providing three-dimensional structures, such as by using two-photon polymerization (2PP), as described, e.g., in Gittard S D et al., “Fabrication of polymer microneedles using a two-photon polymerization and micromolding process,” J. Diabetes Sci. Technol. 2009;3:304-11, which is incorporated by reference in its entirety. Molds of the master can be formed by using a casting technique, in which elastomeric polymers can be used to form molds capable of being removed from an electroplated layer. Additional methods include polymerizing, molding (e.g., melt-molding), spinning, depositing, casting (e.g., melt-casting), etc. Methods of making polymeric structures are described in U.S. Pat. Nos. 7,344,499 and 6,908,453, each of which is incorporated by reference herein in its entirety.

The masters and molds can be formed from any useful material, e.g., a polymer (e.g., such as a biocompatible polymer; an acrylate-based polymer, such as e-Shell 200 (0.5-1.5% wt phenylbis(2,4,6 trimethylbenzoyl)-phosphine oxide photoinitiator, 15-30% wt propylated (2) neopentyl glycoldiacrylate, and 60-80% wt urethane dimethacrylate) or e-Shell 300 (10-25% wt urethane dimethacrylate and 10-20% tetrahydrofurfuryl-2-methacrylate); a resorbable polymer, e.g., polyglycolic acid (PGA), polylactic acid (PLA) including poly(L-lactide) (PLLA) and poly(D-lactide) (PDLA), or PGA-PLA copolymers; or any described herein); an elastomer (e.g., poly(dimethylsiloxane) (PDMS), poly(methylmethacrylate) (PMMA), a hydrogel, as well as including any polymer having a low Young's modulus and a high failure strain); silicon; glass; a metal (e.g., stainless steel, titanium, aluminum, or nickel, as well as alloys thereof); a composite material, etc. In particular embodiments, the master can be formed from any useful material capable of retaining features having micron-scale features (e.g., polymers, metals, silicon, glass, etc.), whereas the mold is formed from an elastomer configured to accurately replicate micron-scale features while also having a low enough Young's modulus to allow removal of the mold by flexing or peeling away the mold from the electroplated layer.

### Microneedle

The present invention includes one or more microneedles of any useful dimension, such as length, width, height, circumference, and/or cross-sectional dimension. In particular, a skilled artisan would be able to optimize the needle length based on the type of fluid or type of tissue to be measured. For instance, the skin can be approximated as two layers including the epidermis (thickness of 0.05 to 1.5 mm) and the dermis (thickness of 0.3 to 3 mm). Accordingly, to obtain fluid in the dermis layer, the needle can be optimized to have a length that is more than about 0.3 mm, 0.5 mm, 1 mm, 1.5 mm, 2 mm, 2.5 mm, or 3 mm, depending on the desired location of the apparatus on the body. A desired cross-sectional dimension can be determined by the skin site to be sampled (e.g., a dimension to allow for local testing of the subject, while minimizing pain), by the desired flow rate of the sample within the lumen of the needle (e.g., the flow rate can be optimized to allow for obtaining a fluid within a particular sampling time, or to minimize sample contamination, coagulation, and/or discomfort to the subject), by the desired volume of sample to be collected, etc.

To access a sample within a subject, each needle can have one or more puncturing edges of any useful geometry. In some embodiments, the puncturing edge at the distal end of the needle includes a tapered point. In particular embodiments, the tapered point is located at the apex of a pyramidal needle, where the base of the needle is attached to the substrate and one side of the pyramidal needle is open, thereby forming the lumen of the needle. An exemplary pyramidal needle is provided in FIG. 2A herein. In yet other embodiments, the puncturing edge is a sharpened bevel for any useful geometrical shape forming the hollow needle, such as a cylinder, a cone, a post, a rectangle, a square, a trapezoid, as well as tapered forms thereof (e.g., a tapered cylinder or a tapered post), etc. In further embodiments, the puncturing edge includes one or more prongs (e.g., two, three, four, five, or more prongs) for obtaining a sample from a subject.

Each microneedle can include one or more orifices that provide fluidic communication with the internal bore of the hollow microneedle. The orifice can have any useful geometry and configuration, such as position along the microneedle. For instance, the orifice can be located at the tip or apex of the microneedle, along a vertical face or wall of the microneedle, at an axis that is off-set from a center axis of the microneedle, at a position that is midpoint between the tip and the base of the needle, at a position that is distanced from the tip of the needle, etc. To provide these orifices, the masters can be designed accordingly.

In some embodiments, the apparatus includes a return needle configured to return tested or analyzed fluid back to the target site. In this way, additional storage chambers will not be needed on-chip to store tested samples. Alternatively, the apparatus can include one or more compartments to maintain tested samples for further or later testing.

The needles can be formed from any useful material, e.g., a seeding material and/or an electroplating material. Exemplary materials include a metal (e.g., stainless steel, titanium, aluminum, nickel, iron, gold, copper, nickel, chromium, or tungsten), alloys thereof (e.g., a nickel-iron alloy), multilayers thereof (e.g., layers of titanium and copper or layers of titanium and gold), or a composite material, etc. The surface (e.g., interior and/or exterior surface) of the needle can be surface-modified with any agent described herein (e.g., a linking agent, capture agent, label, and/or porous material, as described herein). Additional surface-modified needles are described in U.S. Pub. No. 2011/0224515, as well as U.S. Pat. Nos. 7,344,499 and 6,908,453, each of which is incorporated by reference herein in its entirety.

Furthermore, a plurality of needles can be provided in an array. The array can include two, three, four, five, six, seven, eight, nine, ten, fifteen, twenty, or more needles configured in any useful arrangement (e.g., geometrical arrangements). The array can have any useful spatial distribution of needles (e.g., a square, rectangular, circular, or triangular array), a random distribution, or the like.

The needle can include any useful substance, e.g., any described herein. In particular embodiments, one or more needles include a substance that further includes one or more capture agents. For example, the needle can include (e.g., within a portion of the lumen of the needle) a matrix including an electroactive component. The electroactive component can be, e.g., a carbon paste including one or more capture agents (e.g., an enzyme or a catalyst (e.g., rhodium) for detecting a marker). Further embodiments are described in Windmiller J R et al., “Microneedle array-based carbon paste amperometric sensors and biosensors,” Analyst 2011;136:1846-51, which is incorporated by reference in its entirety. Exemplary needles are described in U.S. Pub. No. 2011/0224515; and Int. Pub. No. WO 2013/058879, each of which is incorporated by reference in its entirety.

### Sensors

The microneedles can be interfaced with any useful sensor. Exemplary sensors are described in Miller P R et al., “Hollow microneedle-based sensor for multiplexed transdermal electrochemical sensing,” J. Vis. Exp. 2012 Jun 1;(64):e4067; and Miller P R et al., “Multiplexed microneedle-based biosensor array for characterization of metabolic acidosis,” Talanta 2012 Jan 15;88:739-42, each of which is incorporated herein by reference in its entirety.

The sensor can include one or more transducers, which can be any useful structure for detecting, sensing, and/or measuring a marker or target of interest. Exemplary transducers include one or more of the following: optical sensors (e.g., including measuring one or more of fluorescence spectroscopy, interferometry, reflectance, chemiluminescence, light scattering, surface plasmon resonance, or refractive index), piezoelectric sensors (e.g., including one or more quartz crystals or quartz crystal microbalance), electrochemical sensors (e.g., one or more of carbon nanotubes, electrodes, field-effect transistors, etc.), etc., as well as any selected from the group consisting of an ion selective electrode, an ion sensitive field effect transistor (e.g., a n-p-n type sensor), a light addressable potentiometric sensor, an amperometric sensor (e.g., having a two-electrode configuration (including reference and working electrodes) or a three-electrode configuration (including reference, working, and auxiliary electrodes)), and/or an impedimetric sensor.

In particular embodiments, the transducer is a working electrode having an exposed working area. The working electrode includes any useful conductive material (e.g., gold, indium tin oxide, titanium, and/or carbon). Optionally, the working area is surface modified, e.g., with a linking agent and/or a capture agent described herein. These transducers can include one or more other components that allows for detection, such as a ground electrode, a reference electrode, a counter electrode, a potentiostat, etc. The electrode can have any useful configuration, such as, e.g., a disk electrode, a spherical electrode, a plate electrode, a hemispherical electrode, a microelectrode, or a nanoelectrode; and can be formed from any useful material, such as gold, indium tin oxide, carbon, titanium, platinum, etc.

Exemplary electrodes include a planar electrode, a three-dimensional electrode, a porous electrode, a post electrode, a microelectrode (e.g., having a critical dimension on the range of 1 to 1000 μm, such as a radium, width, or length from about 1 to 1000 μm), a nanoelectrode (e.g., having a critical dimension on the range of 1 to 100 nm, such as a radium, width, or length from about 1 to 100 nm), as well as arrays thereof. For instance, a three-dimensional (3D) electrode can be a three-dimensional structure having dimensions defined by interferometric lithography and/or photolithography. Such 3D electrodes can include a porous carbon substrate. Exemplary 3D porous electrodes and methods for making such electrodes are described in U.S. Pat. No. 8,349,547, which is incorporated herein by reference in its entirety. In another embodiment, the electrode is a porous electrode having a controlled pore size (e.g., a pore size less than about 1 μm or about 0.1 μm). In some embodiments, the electrode is a post electrode that is a carbon electrode (e.g., formed from a photoresist (e.g., an epoxy-based resist, such as SU-8) that has been pyrolyzed), which can be optionally modified by depositing a conductive material (e.g., a conductive polymer or a metal, such as any described herein). In yet other embodiments, the electrode is a nanoelectrode including a nanodisc, a nanoneedle, a nanoband, a nanoelectrode ensemble, a nanoelectrode array, a nanotube (e.g., a carbon nanotube), a nanopore, as well as arrays thereof. Exemplary nanoelectrodes are described in Arrigan D W M, “Nanoelectrodes, nanoelectrode arrays and their applications,” Analyst 2004 Dec;129(12):1157-65, which is incorporated by reference herein in its entirety.

Any of these electrodes can be further functionalized with a conductive material, such as a conductive polymer, such as any described herein, including poly(bithiophene), polyaniline , or poly(pyrrole), such as dodecylbenzenesulfonate-doped polypyrrole; a metal, such as metal nanoparticles (e.g., gold, silver, platinum, and/or palladium nanoparticles), metal microparticles, a metal film (e.g., palladium or platinum), etc.; a nanotube; etc. Additional electrodes are described in Int. Pub. No. WO 2013/058879 and U.S. Pat. No. 8,349,547, each of which is incorporated herein by reference in its entirety.

The needles and transducers can be configured in any useful manner. For instance, the needles and transducers can be fluidically connected by a fluidic channel. In other embodiments, the needle can include a transducer within the lumen of a needle, such as those described in Int. Pub. No. WO 2013/058879, which is incorporated by reference in its entirety. In some embodiments, the needle can include a transducer on the exterior surface of the needle. For instance, the transducer can include one or more conductive layers on the exterior surface of the needle, where the conductive layer can include one or more capture agents (e.g., any described herein). Such needles and conductive layers, as well as sensing layers and protective layers, are described in, e.g., Int. Pub. No. WO 2006/116242, which is incorporated herein by reference in its entirety.

The transducer can be integrated with the needle by any useful process and with any useful configuration. For example, the transducer can be a carbon fiber electrode configured to reside within the lumen of a needle. Such a configuration is described, e.g., in Miller P R et al., “Integrated carbon fiber electrodes within hollow polymer microneedles for transdermal electrochemical sensing,” Biomicrofluidics 2011;5:013415 (14 pages), which is incorporated herein by reference in its entirety.

The present invention could also allow for integration between one or more needles with an array of transducers. The needle and electrode can be configured in any useful way. For instance, each needle can be associated with a particular electrode, such that there is a one-to-one correspondence between the fluid withdrawn into the needle and the fluid being delivered to the electrode. In other embodiments, each needle is associated with an array of electrodes. In yet other embodiments, an array of needles is associated with an individual electrode or with an array of electrodes.

The fluidic connection between the needle and the electrode can be established by a channel or a network of channels. In one non-limiting example, when one needle is associated with an array N×M of electrodes, a network containing channels can be interfaced between the needle and electrode array. Such a network can include a main channel that splits into N sub-channels, which in turn split into M smaller channels. A skilled artisan would understand how to optimize channel geometry to fluidically connect one or more needles to one or more electrodes.

In some embodiments, the array is a high density array including N×M array of electrodes, where each electrode can be individually addressable. In further embodiments, the high density array is surface modified with one or more capture agents and/or one or more linking agents, as described herein. Exemplary values for N and M include, independently, 1, 2, 3, 4, 5, 6, 7, 8, 9, 10, 12, 15, 20, 25, 30, 40, 50, 75, 100, etc.

The transducers can optionally be surface-modified with one or more capture agents (e.g., one or more antibodies for detecting one or more markers, such as any described herein). Such transducer can include, e.g., an ion selective electrode (ISE) for detecting one or more ions. An ISE can include a porous material and one or more capture agents, such as, e.g., one or more ionophores. Exemplary porous materials include porous carbon, graphene, silicon, conducting polymer (e.g., such as any described herein), etc. Exemplary ionophores include one or more of the following: a crown ether, a macrocyclic compound, a cryptand, a calixarene, A23187 (for Ca²⁺), beauvericin (for Ca²⁺, Ba²⁺), calcimycine (for A23187), enniatin (for ammonium), gramicidin A (for H⁺, Na⁺, K⁺), ionomycin (for Ca²⁺), lasalocid, monensin (for Na⁺, H-), nigericin (for K⁺, H⁺, Pb⁺), nonactin (for ammonium), nystatin, salinomycin (for K⁺), valinomycin (for K⁺), siderophore (for Fe³⁺), etc. Such materials and ISEs can be obtained by any useful process, such as templating (see, e.g., Lai C et al., Anal. Chem. 2007;79:4621-6), interference lithography, molding, casting, spinning, electrospinning, and/or depositing.

Another exemplary transducer includes a detection electrode configured for a sandwich assay. Such an electrode include, e.g., a conductive surface and a first capture agent (e.g., an antibody) immobilized on the conductive surface, where the first capture agent is optionally attached by a linking agent. In use, the marker of interest binds to the first capture agent to form a complex, and further capture agents can be used to bind the resultant complex. To detect the complex, further capture agents can include a detectable label or an enzyme that reacts with an agent to provide a detectable signal (e.g., an agent that is a fluorogenic, enzyme-cleavable molecule).

### Fluidic Channels, Chambers, and Depots

One or more fluidic channels (including inlets), chambers, and depots can be used to effect fluidic communication between two structures or regions. In particular embodiments, depots are fluidic chambers configured to store one or more therapeutic agents.

The present invention could also allow for integration between one or more needles with an array of depots. For instance, each needle can be associated with a particular depot, such that there is a one-to-one correspondence between the type of therapeutic agent being injected into the user and one particular needle. In other embodiments, each needle is associated with an array of depots. In yet other embodiments, an array of needles is associated with an individual depot or with an array of depots. The fluidic connection between the needle and the depots can be established by a channel or a network of channels.

Any of the fluidic channels, chamber, and depots described herein can be surface modified (e.g., to increase biocompatibility, decrease protein adsorption or absorption, and/or decrease surface contamination). Furthermore, such fluidic channels, chamber, and depots can also include one or more capture agents to selectively or non-selectively bind to cellular components or contaminants within a sample.

### Surface Modification

Any of the surfaces described herein may be modified to promote biocompatibility, to functionalize a surface (e.g., using one or more capture agents including the optional use of any linking agent), or both. Exemplary surfaces include those for one or more transducers, needles, fluidic channels, depots, filters, and/or substrates (e.g., a PCB substrate).

The surface can be modified with any useful agent, such as any described herein. Exemplary agents include a capture agent (e.g., any described herein, such as an antibody); a polymer, such as a conducting polymer (e.g., poly(pyrrole), poly(aniline), poly(3-octylthiophene), or poly(thiophene)), an antifouling polymer, or a biocompatible polymer (e.g., chitosan), or a cationic polymer)); a coating, e.g., a copolymer, such as a copolymer of an acrylate and a lipid, such as butyl methacrylate and 2-methacryloyloxyethyl phosphorylcholine; a film; a label (e.g., any described herein); a linking agent (e.g., any described herein); an electroactive component, such as one or more carbon nanotubes or nanoparticles (e.g., gold, copper, cupric oxide, silver, or platinum nanoparticles), such as, for stabilizing an electrode; an enzyme, such as glucose oxidase, cholesterol oxidase, horse radish peroxidase, or any enzyme useful for oxidizing, reducing, and/or reacting with a marker of interest; or combinations thereof (e.g., an electroactive component coated with a polymer, such as a carbon nanotube coated with polyaniline).

Optionally, linking agents can be used be attach the agent to the surface. Exemplary linking agents include compounds including one or more first functional groups, a linker, and one or more second functional groups. In some embodiments, the first functional group allows for linking between a surface and the linker, and the second functional group allows for linking between the linker and the agent (e.g., a capture agent, a label, or any agent described herein). Exemplary linkers include any useful linker, such as polyethylene glycol, an alkane, and/or a carbocyclic ring (e.g., an aromatic ring, such as a phenyl group). In particular embodiments, the linking agent is a diazonium compound, where the first functional group is a diazo group (—N₂), the linker is an aryl group (e.g., a mono-, bicyclic, or multicyclic carbocyclic ring system having one or two aromatic rings and is exemplified by phenyl, naphthyl, xylyl, 1,2-dihydronaphthyl, 1,2,3,4-tetrahydronaphthyl, fluorenyl, indanyl, indenyl, and the like), and the second functional group is a reactive group for attaching a capture agent or a label (e.g., where the second functional group is halo, carboxyl, amino, sulfo, etc.). Such diazonium compounds can be used to graft an agent onto a surface (e.g., an electrode having a silicon, iron, cobalt, nickel, platinum, palladium, zinc, copper, or gold surface). In some embodiments, the linking agent is a 4-carboxybenzenediazonium salt, which is reacted with a capture agent by 1-ethyl-3-[3-dimethylaminopropyl]carbodiimide hydrochloride (EDC)/N-hydroxy succinimide (NETS) crosslinking, to produce a diazonium-capture agent complex. Then, this resultant complex is deposited or grafted onto a surface (e.g., an electrode surface). Other exemplary linking agents include pairs of linking agents that allow for binding between two different components. For instance, biotin and streptavidin react with each other to form a non-covalent bond, and this pair can be used to bind particular components.

### Additional Components

The present microneedles and arrays can be included in an apparatus having any useful additional component. Exemplary components include those provided for a transducer (e.g., any described herein, as well as those in Justino C I L et al., “Review of analytical figures of merit of sensors and biosensors in clinical applications,” Trends Analyt. Chem. 2010;29:1172-83, which is incorporated by reference in its entirety); those provided for a microneedle (e.g., any described herein, as well as those in Gittard S D et al., “Two photon polymerization of microneedles for transdermal drug delivery,” Exp. Opin. Drug Deliv. 2010;7(4):513-33, and Miller P R et al., “Multiplexed microneedle-based biosensor array for characterization of metabolic acidosis,” Talanta 2012;88:739-42, each of which is incorporated by reference in its entirety); a membrane (e.g., placed between the needle and the channel; placed within a channel, such as to filter one or more particles within the sample; and/or placed between the channel and the electrode); a multifunctional sensor (e.g., to measure temperature, strain, and electrophysiological signals, such as by using amplified sensor electrodes that incorporate silicon metal oxide semiconductor field effect transistors (MOSFETs), a feedback resistor, and a sensor electrode in any useful design, such as a filamentary serpentine design); a microscale light-emitting diode (LEDs, such as for optical characterization of the test sample); an active/passive circuit element (e.g., such as transistors, diodes, and resistors); an actuator; a wireless power coil; a device for radio frequency (RF) communications (e.g., such as high-frequency inductors, capacitors, oscillators, and antennae); a resistance-based temperature sensor; a photodetector; a photovoltaic cell; and a diode, such as any described in Kim Net al., Science 2011;333:838-43, which is incorporated herein by reference. These components can be made from any useful material, such as, e.g., silicon and gallium arsenide, in the form of filamentary serpentine nanoribbons, micromembranes, and/or nanomembranes.

The apparatus can include one or more structural components within the integral platform or substrate. Exemplary components include a mixing chamber in fluidic communication with the lumen of a needle; a reservoir optionally including one or more reagents (e.g., any described herein), where the reservoir can be in fluidic communication with the mixing chamber or any fluidic channel; a cell lysis chamber (e.g., configured to lyse one or more cells in a sample and in fluidic communication with needle and the sensing transducer); a controllable valve (e.g., configured to release a reagent from a reservoir into a mixing chamber); a pump (e.g., configured to facilitate flow of a sample to the transducer and/or through one or more fluidic channels); a waste chamber (e.g., configured to store a sample after detection of one or more reagents); a probe; and/or a filter (e.g., configured to separate one or more components from the sample either before or after detection with the transducer).

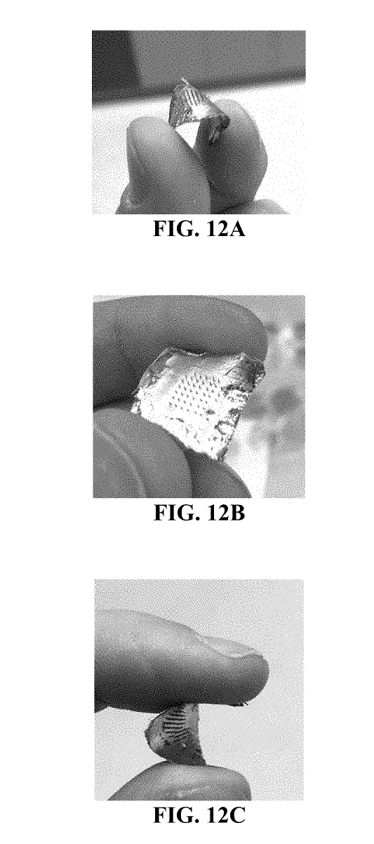

In some embodiments, the needle can be configured to be in fluidic communication with a reservoir (e.g., containing a drug for delivery and/or a reagent for detecting the marker of interest). Such a configuration can optionally include a valve between the needle and reservoir. In other embodiments, a probe can be configured to be in fluidic communication with the lumen of the needle. Exemplary needles and probes are described in Int. Pub. No. WO 2013/058879 (e.g., in FIG. 1A-1D, FIG. 1L, FIG. 2A-2C, FIG. 5A-5D, FIG. 12A-12B, FIG. 17, FIG. 18A-18D, and its related text), which is incorporated herein in its entirety.

The apparatus can include one or more components to operate a transducer. For instance, in some embodiments, the transducer is an electrode or an array of electrodes. Accordingly, the apparatus can further include a power source to operate the electrode. In particular embodiments, the apparatus includes a data-processing circuit powered by the power source and electrically connected to the transducer (e.g., a counter electrode, a reference electrode, and at least one said working electrode). In further embodiments, the apparatus includes a data output port for the data-processing circuit. Such data from the transducer can include any useful information, such as electromotive force (EMF), potentiometric, amperometric, impedance, and/or voltammetric measurements. Other data can include fluorometric, colorimetric, optical, acoustic, resonance, and/or thickness measurements.

An apparatus can be provided in any useful package. For instance, such a package can include a packaged chip having a housing for the apparatus of the invention. In one embodiment, the housing includes a substantially planar substrate having an upper surface and an opposing lower surface; a first fluidic opening disposed on the upper surface of the substrate; a second fluidic opening disposed on the lower surface of the substrate; a first fluidic channel fluidically connecting the first fluidic opening to the second fluidic opening; and a first adhesive layer adhered to the upper surface, having a hole disposed through the layer, wherein the hole is substantially aligned with, and fluidically coupled to, the first fluidic opening in the substrate. In some embodiments, the housing includes one or more structures allowing for integrating with a fluidic printed wiring board having a standard electrical printed circuit board and one or more fluidic channels embedded inside the board. An exemplary packaged chip is provided in U.S. Pat. No. 6,548,895, which is incorporated by reference in its entirety. Further components for a packaged chip include a substrate including an electrically insulating material, one or more electrical leads, a substantially planar base, an external fixture, etc., as well as any other components described in U.S. Pat. Nos. 6,443,179 and 6,548,895, each of which is incorporated herein by reference in its entirety.

The apparatus of the invention can be provided in any useful format. For instance, the apparatus can be provided with particular components integrated into one package or monolithic structure. A non-limiting integrated apparatus can include an array of microneedles, fluidics, and electrode array that are provided in an integrated format. In other examples, the apparatus is provided as a modular package, in which the needles, fluidics, and electrodes are provided as separate plug-and-play modules that can be combined. In particular embodiments, a sensor module includes a packet of electrode arrays with each packet containing specific chemistries. In further embodiments, the sensor module is configured to be relevant for the desired analyte, such as to detect a particular drug or a particular virus. Further modules can include a needle module including one or more needles (e.g., an array of needles); a fluidics module including one or more chambers, valves, and/or channels; a delivery module including one or more therapeutic agents; and/or a reagent module including one or more prepackaged reagents and buffers configured for a particular test or analyte. Such modules can be reusable or disposable. For instance, if the sample processing is extensive, one would want a reusable fluidics module, which is configured for fluidic communication with the needle module and sensor module. In further embodiments, the needle and sensor modules can be disposable. In another example, if sample processing or sensing requires an elaborate needle (e.g., a needle having a particular geometrical configuration and/or surface modification), then the needle module can be configured to be reusable. Other considerations include possibility of contamination of one or more modules, etc. A skilled artisan would understand how modules can be configured for fluidic communication with other modules and designed for reusability or disposability.

The present invention can be useful for autonomous remote monitoring of a subject. The apparatus of the invention can be placed on the skin of a subject, and the presence or absence of one or more markers can be remotely relayed to a heath care worker. Accordingly, the apparatus described herein can include one or more components that would allow for such relay. Exemplary components include an analog-to-digital converter, a radiofrequency module, and/or a telemetry unit (e.g., configured to receive processed data from a data-processing circuit electrically connected to the transducer and to transmit the data wirelessly). In various embodiments, the telemetry unit is fixed within the platform or packaged separately from the platform and connected thereto by a cable.

### Multiple Reactions

The present microneedles, as well as any apparatus including such microneedles, can be used to perform multiple reactions on-chip. Such reactions can include those to prepare a sample (e.g., to dilute, concentrate, or filter a sample), to bind the sample to a capture agent, to prepare one or more reagents to be reacted with the sample (e.g., to reconstitute a reagent on-chip prior to reacting with the sample), to react the sample with any useful reagent, to store the sample on-chip, and/or to perform other post-processing reactions. To perform multiple reactions, the microneedles, fluidic channels, and transducers can be provided in an array format, such as any described herein.

To allow for multiple reactions or processing steps, the apparatus can include additional chambers in fluidic communication with one or more needles. In one embodiment, the apparatus includes one or more mixing chambers in fluidic communication with one or more needles and configured to receive the sample or a portion thereof. The mixing chamber can include one or more reagents (e.g., any described herein), buffers, diluents (e.g., water or saline), salts, etc. Optionally, the mixing chamber can include one or more components to assist in mixing, such as one or more of the following: a bead, a passive mixer, a rotary mixer, a microbubble, an electric field to induce electrokinetic and/or dielectrophoretic flow, a staggered structure to induce chaotic advection, an acoustic mixer, a heater to induce a thermal gradient, and/or a magnetic bead for use with a magnetic field generator.

The apparatus can also include one or more reaction chambers (e.g., to combine one or more reagents (e.g., one or more enzymes and/or beads) within this chamber and/or to incubate reaction mixtures including the sample or a portion thereof), lysing chambers (e.g., to lyse one or more cells within the sample), washing chambers (e.g., to wash one or more components within the sample), elution or extraction chambers (e.g., including one or more filters, particles, beads, sieves, or powders to extract one or more components from the sample), and/or collection chambers (e.g., to collect one or more processed samples or aliquots thereof). In particular embodiments, at least one reaction chamber is in fluidic communication with at least one mixing chamber by a channel. In further embodiments, the reaction chamber is in fluidic communication two or more mixing chambers, thereby combining the substance in each mixing chamber within the reaction chamber. In this manner, parallel or serial sequences of substances can be combined in a controlled manner within a reaction chamber or multiple reaction chambers. A skilled artisan would be able to design arrays of mixing and/or reaction chambers (optionally interconnected with channels) to effect the proper sequence of each reaction step.

Any of the chambers and channels interconnecting such chambers can be surface modified, as described herein. Furthermore, such chambers and channels can include further structures that would be useful for detecting one or more markers. For instance, one or more filters or membranes can be used to separate particular components from the sample and/or the reaction mixture. For instance, when the sample is whole blood, a filter can be used to separate the plasma from other blood components, such as the red blood cells.

### Test Samples

The present microneedles can be used to access and/or test any useful test sample, such as blood (e.g., whole blood), plasma, serum, transdermal fluid, interstitial fluid, sweat, intraocular fluid, vitreous humor, cerebrospinal fluid, extracellular fluid, lacrimal fluid, saliva, mucus, etc., and any other bodily fluid.

The sample can be obtained from any useful source, such as a subject (e.g., a human or non-human animal), a plant (e.g., an exudate or plant tissue, for any useful testing, such as for genomic and/or pathogen testing), an environment (e.g., a soil, air, and/or water sample), a chemical material, a biological material, or a manufactured product (e.g., such as a food or drug product).

### Substances, Including Reagents and Therapeutic Agents

The present apparatus can further be adapted to deliver one or more substances from a reservoir to another region of the apparatus or to a subject. In some embodiments, the apparatus includes one or more reservoirs including a substance for detecting one or more markers of interest. Exemplary substances include a reagent (e.g., any described herein, such as a label, an antibody, a dye, a capture agent, etc.), a buffer, a diluent, a salt, etc.

In other embodiments, the apparatus includes one or more substances that can be injected or delivered to a subject (e.g., one or more therapeutic agents). Such therapeutic substances include, e.g., an analgesic, anesthetic, antiseptic, anticoagulant, drug (e.g. adrenaline and/or insulin), vaccine, medical countermeasure, etc.

### Capture Agents and Labels

Any useful capture agents and labels can be used in combination with the present invention. The capture agent can directly or indirectly bind the marker of interest. The label can be used to directly or indirectly detect a marker. For direct detection, the label is conjugated to a capture agent that binds to the marker. For instance, the capture agent can be an antibody that binds the marker, and the label for direct detection is a nanoparticle attached to the capture agent. For indirect detection, the label is conjugated to a second capture agent that further binds to a first capture agent. A skilled artisan would understand how to optimize combinations of labels, capture agents, and linking agents to detect a marker of interest.

Further, multiple capture agents can be used to bind the marker and provide a detectable signal for such binding. For instance, multiple capture agents are used for a sandwich assay, which requires at least two capture agents and can optionally include a further capture agent that includes a label allowing for detection.

Exemplary capture agents include one or more of the following: a protein that binds to or detects one or more markers (e.g., an antibody or an enzyme), a globulin protein (e.g., bovine serum albumin), a peptide, a nucleotide, a nanoparticle, a microparticle, a sandwich assay reagent, a catalyst (e.g., that reacts with one or more markers), and/or an enzyme (e.g., that reacts with one or more markers, such as any described herein). The capture agent can optionally include one or more labels, e.g. any described herein. In particular embodiments, more than one capture agent, optionally with one or more linking agents, can be used to detect a marker of interest. Furthermore, a capture agent can be used in combination with a label (e.g., any described herein) to detect a maker. Exemplary labels include one or more fluorescent labels, colorimetric labels, quantum dots, nanoparticles, microparticles, barcodes, radio labels (e.g., RF labels or barcodes), avidin, biotin, tags, dyes, an enzyme that can optionally include one or more linking agents and/or one or more dyes, as well as combinations thereof etc.

### Markers, Including Targets

The present microneedles, as well as apparatus including such microneedles, can be used to determine any useful marker or targets. Exemplary markers include one or more physiologically relevant markers, such as glucose, lactate, pH, a protein (e.g., myoglobin, troponin, insulin, or C-reactive protein), an enzyme (e.g., creatine kinase), a catecholamine (e.g., dopamine, epinephrine, or norepinephrine), a cytokine (e.g., TNF-α or interleukins, such as IL-6,IL-12, or IL-1(3), an antibody (e.g., immunoglobulins, such as IgA), a biomolecule (e.g., cholesterol or glucose), a neurotransmitter (e.g., acetylcholine, glutamate, dopamine, epinephrine, neuropeptide Y, or norepinephrine), a signaling molecule (e.g., nitric oxide), an antigen (e.g., CD3, CD4, or CD8), an ion (e.g., a cation, such as K⁺, Na⁺, H⁺, or Ca²⁺, or an anion, such as Cl⁻ or HCO₃⁻), CO₂, O₂, H₂O₂, a cancer biomarker (e.g., human ferritin, carcinoembryonic antigen (CEA), prostate serum antigen, human chorionic gonadotropin (hCG), diphtheria antigen, or C-reactive protein (CRP)), a hormone (e.g., hCG, epinephrine, testosterone, human growth hormone, epinephrine (adrenaline), thyroid hormone (e.g., thyroid-stimulating hormone (TSH), thyroxine (TT4), triiodothyronine (TT3), free thyroxine (FT4), and free triiodothyronine (FT3)), adrenal hormone (e.g., adrenocorticotrophic hormone (ACTH), cortical hormone (F), and 24-hour urine-free cortisol (UFC)), a gonadal hormone (e.g., luteinizing hormone (LH), follicle-stimulating hormone (FSH), testosterone, estradiol (E2), and prolactin (PRL)), cortisol, leptin, or a peptide hormone, such as insulin), an inflammatory marker (e.g., CRP), a disease-state marker (e.g., glycated hemoglobin for diabetes or markers for stress or fatigue), a cardiovascular marker (e.g., CRP, D-dimer, troponin I or T), a blood marker (e.g., hematocrit, or hemoglobin), a cell (e.g., a leukocyte, neutrophil, B-cell, T-cell, lymphocyte, or erythrocyte), a viral marker (e.g., a marker for human immunodeficiency virus, hepatitis, influenza, or chlamydia), a metabolite (e.g., glucose, cholesterol, triglyceride, creatinine, lactate, ammonia, ascorbic acid, peroxide, potassium, glutamine, or urea), a nucleic acid (e.g., DNA and/or RNA for detecting one or more alleles, pathogens, single nucleotide polymorphisms, mutations, etc.), an amino acid (e.g., glutamine), a drug (e.g., a diuretic, a steroid, a growth hormone, a stimulant, a narcotic, an opiate, etc.), etc. Other exemplary markers include one or more pathogens, such as Mycobacterium tuberculosis, Diphtheria antigen, Vibrio cholera, Streptococcus (e.g., group A), etc.

In particular embodiments, the marker is indicative of exhaustion (e.g., exercise-induced exhaustion) and/or fatigue (e.g., severe fatigue, such as in deployed military personnel). Such markers include, e.g., ACTH, ascorbic acid, CD3, CD4, CD8, CD4/CD8, cholesterol, cortical hormone, cortisol, creatine kinase, E2, epinephrine, FSH, FT3, FT4, glucose, glutamine, glutamate, hematocrit, hemoglobin, human growth hormone, IgA, insulin, insulin-like growth factor, interleukin-6, iron, lactate (e.g., serum or blood lactate), leptin, LH, neuropeptide Y, norepinephrine, peroxide, pH, potassium, PRL, TSH, TT3, TT4, testosterone, and/or urea.

### Methods and Use

The present microneedles can be applied for any useful method and/or adapted for any particular use, apparatus, or device. For instance, point-of-care (POC) diagnostics allow for portable systems, and the apparatus herein can be adapted for POC use. In some embodiments, the apparatus for POC use includes a test sample chamber, a microfluidic processing structure (e.g., any structure described herein, such as a needle, a substrate, and/or a channel), a target recognition region (e.g., including any transducer described herein), an electronic output, a control (e.g., a positive and/or negative controls), and/or a signal transduction region. Exemplary POC apparatuses and uses are described in Gubala V et al., “Point of care diagnostics: status and future,” Anal Chem. 2012;84(2):487-515, which is incorporated by reference in its entirety. Such POC apparatuses can be useful for detecting one or more markers for patient care, drug and food safety, pathogen detection, diagnostics, etc.

Wearable sensors are a new paradigm in POC apparatuses, allowing for minimally invasive monitoring of physiological functions and elimination of biological fluid transfer between subject and apparatus; these apparatuses can be capable of providing real-time analysis of a patient's condition. In other embodiments, the apparatus is adapted to include one or more components allowing for a wearable sensor. Exemplary wearable sensors, as well as relevant components, are described in Windmiller J R et al., “Wearable electrochemical sensors and biosensors: A review,” Electroanalysis 2013;25:29-46. Such components include a telemetry network including one or more apparatuses (e.g., as described herein), one or more flexible substrates (e.g., where one or more transducers are integrated into a flexible substrate, such as cloth, plastic, or fabric, e.g., Gore-Tex®, an expanded polytetrafluoroethylene (ePTFE), polyimide, polyethylene naphthalate, polyethylene terephthalate, biaxially-oriented polyethylene terephthalate (e.g., Mylar®), or PTFE), and/or one or more flexible electrodes (e.g., a screen printed electrode printed on a flexible substrate, such as any herein).

In some embodiments, the apparatus of the invention is adapted as an epidermal electronic device. Such devices can include, e.g., one or more printed flexible circuits that can be stretched and bent to mimic skin elasticity can perform electrophysiological measurements such as measuring temperature and hydration as well as monitoring electrical signals from brain and muscle activity. Exemplary components for such a device are described in Kim N et al., Science 2011;333:838-43, which is incorporated herein by reference.

In other embodiments, the apparatus of the invention is adapted as a temporary tattoo. Such tattoos can include, e.g., one or more screen printed electrodes directly attached to the skin were recently reported to measure lactate through sweat. Exemplary components for such an apparatus are described Jia W et al., “Electrochemical tattoo biosensors for real-time noninvasive lactate monitoring in human perspiration,” Anal. Chem. 2013;85:6553-60, which is incorporated herein by reference.

The apparatus of the invention can be configured for any useful method or treatment. For instance, the apparatus can be configured for locally treating, delivering, or administering a therapeutic substance after detecting one or more markers. Exemplary methods and apparatuses are described in Int. Pub. No. WO 2010/022252, which is incorporated herein by reference.

### EXAMPLES

### Example 1

### Flexible Hollow Microneedle Arrays Fabricated with an Electromolding Method

Wearable sensors have gone from a next generation idea to commercially available devices for monitoring everything from sleep patterns to daily activity (see, e.g., Takacs J et al., “Validation of the Fitbit One activity monitor device during treadmill walking,” J. Sci. Med. Sport 2014 Sep;17(5):496-500). While these devices are largely limited to measuring vital signs, microneedles are an emerging wearable technology for analyzing physiological changes within the body, both locally and systemically.

Microneedle technology was initially developed for transdermal drug delivery but is becoming an attractive means for transdermal sensing due to its unique ability to acquire interstitial fluid without imposing significant pain to the user (see, e.g., Kim Y C et al., “Microneedles for drug and vaccine delivery,” Adv. Drug Deliv. Rev. 2012 Nov;64(14):1547-6; and El-Laboudi A et al., “Use of microneedle array devices for continuous glucose monitoring: a review,” Diabetes Technol. Ther. 2013 Jan;15(1):101-15). A variety of microneedle designs have been used for sensing applications. In particular, hollow microneedles overcome limitations of fouling that are potentially seen with solid microneedles and have been incorporated into a device to continuously measure glucose in human subjects for 72 hours (see, e.g., Invernale M A et al., “Microneedle electrodes toward an amperometric glucose-sensing smart patch,” Adv. Healthc. Mater. 2014 Mar;3(3):338-42; and Jina A et al., “Design, development, and evaluation of a novel microneedle array-based continuous glucose monitor,” J. Diabetes Sci. Technol. 2014 May;8(3):483-7).

While hollow microneedles offer unique benefits compared to other microneedles types for sensing applications, such hollow microneedles pose a greater fabrication challenge than solid microneedles. For instance, solid microneedles are easier to fabricate due to their simpler geometry, but more sophisticated systems are necessary for creating hollow microneedles, which in turn increases cost. Recently, Norman et al. suggested a set of parameters for making hollow microneedles that accounts for their fabrication challenges, cost of materials and equipment, and required microneedle geometry for suitable performance (see, e.g., Norman J J et al., “Hollow microneedles for intradermal injection fabricated by sacrificial micromolding and selective electrodeposition,” Biomed. Microdevices 2013 Apr;15(2):203-10). Additionally, not all hollow microneedles designs perform equally; and improved performance has been shown for designs providing a bore that is off-set from the tip (see, e.g., Mukerjee E V et al., “Microneedle array for transdermal biological fluid extraction and in situ analysis,” Sens. Actuat. A 2004;114(2-3):267-75). Asymmetrical bore placement, e.g., placement that is not concentric about the tip or along the center axis of the microneedle, provides additional fabrication challenges

A variety of techniques exist for making hollow microneedle arrays from a range of different materials, each with its own inherent strengths and weaknesses. The first hollow microneedle arrays were made from standard silicon microfabrication techniques, which also pioneered the first solid microneedles, with either an in-plane or out of place configuration (see, e.g., Gardeniers H J G E et al., “Silicon micromachined hollow microneedles for transdermal liquid transport,” J. Microelectromech. Sys. 2003;12(6):855-62; Paik S J et al., “In-plane single-crystal-silicon microneedles for minimally invasive microfluid systems,” Sens. Actuat. A 2004;114(2-3):276-8; and Henry S et al., “Microfabricated microneedles: a novel approach to transdermal drug delivery,” J. Pharm. Sci. 1998 Aug;87(8):922-5). While these methods created microneedles with an offset bore placement, the resulting structures were brittle, expensive, and had some limitations of control over the desired geometry.

Since that time, a few other stand-alone fabrication methods were used for microneedle fabrication. Microstereolithography was used for creation of hollow microneedles and dozens of arrays could be fabricated within a single batch. Nonetheless, resolution and material compatibility limit this technique (see, e.g., Miller P R et al., “Integrated carbon fiber electrodes within hollow polymer microneedles for transdermal electrochemical sensing,” Biomicrofluidics 2011 Mar 30;5(1):1341).

Recently, Yung et al. used micro-injection molding for creation of hollow microneedles arrays made from a polymer (see, e.g., Yung K L et al., “Sharp tipped plastic hollow microneedle array by microinjection moulding,” J. Micromech. Microeng. 2012;22(1):015016). Injection molding is an industrial fabrication technique for scalable production of parts. Lack of tip sharpness and control of microneedle geometry still limits this fabrication system.

Due to the limitations of these techniques, multiple groups have sought new methods for making hollow microneedles using a combination of fabrication methods. For instance, Davis et al. created hollow microneedles via electroplating into polymer substrates containing conical voids made via laser micromachining (see, e.g., Davis S P et al., “Hollow metal microneedles for insulin delivery to diabetic rats,” IEEE Trans. Biomed. Eng. 2005;52(5):909-15). Upon selectively dissolving the polymer, arrays of metal hollow microneedles were used to deliver insulin to diabetic rats. Other groups have used a similar technique for electroplating into predefined voids or over solid microneedles that were later cut for creation of the bore (see, e.g., Kim K et al., “A tapered hollow metallic microneedle array using backside exposure of SU-8,” J. Micromech. Microeng. 2004;14(4):597-60; and Lee K et al., “Drawing lithography: three-dimensional fabrication of an ultrahigh-aspect-ratio microneedle,” Adv. Mater. 2010 Jan 26; 22(4):483-6). While these techniques all create hollow microneedles, such techniques limited in their ability to create an off-set bore with a flexible substrate.

Due to the limitations with fabricating hollow microneedles, some groups have used solid microneedles made from hydrogels in order to extract fluid for analysis from the skin of both rats and humans (see, e.g., Donnelly R F et al., “Hydrogel-forming microneedles increase in volume during swelling in skin, but skin barrier function recovery is unaffected,” J. Pharm. Sci. 2014 May;103(5):1478-86; and Romanyuk A V et al., “Collection of analytes from microneedle patches,” Anal. Chem. 2014 Nov. 4;86(21):10520-3). These devices do not require a particular geometry to function since the material naturally wicks fluid upon contact. The hydrogel offered a high degree of biocompatibility, and microneedle strength can be tailored by selecting particular polymer precursors in order to improve insertion into the skin. Such solid hydrogel microneedles provided limited volumes of extracted fluids and may be difficult to integrate into sensor systems.

A few groups have also explored a molding methodology for creating hollow microneedles that lend towards scalability and facile production. Wang et al. created arrays of hollow microneedles by UV-curing a polymer (photoresist SU-8) into predefined PDMS molds, thereby creating syringe style needles (see, e.g., Wang P C et al., “Hypodermic-needle-like hollow polymer microneedle array using UV lithography into micromolds,” IEEE 24th International Conference on Micro Electro Mechanical Systems (MEMS), held on 23-27 Jan. 2011 in Cancun, Mexico (pp. 1039-42)). The tips of the molds were slanted; and multiple masks were used to selectively fabricate the bore. Arrays were inserted into ex vivo porcine skin and were capable of injecting a dye into the skin.

In another approach, Matteucci et al. used a technique for making hollow polymeric microneedles with offset bores via a molding method that allowed the master to be reused up to 10 times (see, e.g., Pérennès F et al., “Sharp beveled tip hollow microneedle arrays fabricated by LIGA and 3D soft lithography with polyvinyl alcohol,” J. Micromech. Microeng. 2006;16(3):473-9; and Matteucci M et al., “Poly vinyl alcohol re-usable masters for microneedle replication,” Microelectron. Eng. 2009;86(4-6):752-6). In this technique, inverse microneedle masters were made with a pillar rising from within each microneedle mold. When the molds were filled with a PMMA copolymer, the pillars blocked a portion of the mold that formed the microneedle bore. This method did require a sanding step in order to remove excess material to form a continuous lumen. Insertion tests, mechanical characterization, flexibility of the array, and biocompatibility have yet to be performed for microneedles formed in this manner.

Based on the limitations of previous techniques, we investigated a method for fabricating hollow metal microneedle arrays on a flexible substrate from reusable masters and reusable molds with an off-set bore. Such arrays can be useful for sensing applications, where collecting sufficient fluid volume (in a limited time frame) from a single microneedle can be difficult for sensing purposes (see, e.g. Zimmermann S et al., “A microneedle-based glucose monitor: fabricated on a wafer-level using in-device enzyme immobilization,” 12th International Conference on TRANSDUCERS, Solid-State Sensors, Actuators and Microsystems, held on 8-12 Jun. 2003 in Boston, Mass. (vol. 1, pp. 99-102)).

In addition, the methods herein can optionally provide microneedles arrays having some degree of flexibility. Such arrays can conform to the surface of the skin, thereby facilitating extraction or injection at a transdermal site. One exemplary method employs electroplating to control the mechanical and elemental properties of the resulting microneedle and its substrate. For instance, electroplating conditions may be optimized to provide the desired layer thickness for that particular electroplated metal or metal alloy. In addition, electroplating into a defined cavity generally retains the fidelity of the mold, such that the penetrating edges of the microneedle are maintained and are not dulled. The arrays can also be fabricated from materials that possess a suitable level of biocompatibility, which can be important for long-term use of these devices (e.g., long-term residence of these devices for use a wearable sensor). Additional details are provided in the following Examples.

### Example 2

### Fabrication and Testing Methods for Masters, Molds, and Electromolded Hollow Microneedles

Master structure fabrication: Master structures were designed in SolidWorks® as a four sided-pyramid measuring 550 μm in height and 250 μm in its base. An inward facing ledge was placed on one face of the pyramid shaped microneedles for creating an orifice at the distal end of the microneedle bore. Ledges sizes were adjusted in terms of their depth within the microneedle; and ledge sizes ranged from 20 μm to 60 μm. The back wall for the 20 μm ledge was perpendicular to the microneedle substrate, and ledges smaller than this were not expected to sufficiently block the initial seed layer for creating the bore. Thus, ledges having a depth that is less than 20 μm were not tested.

Dimensions of the ledge cutout were maintained at 60 μm (width) and 50 μm (height), while the ledge depth was adjusted. Master structures were fabricated using a two-photon lithography system employing laser direct write. SolidWorks® files were converted to STL files and sliced with a 10 μm step height and 1.5 μm raster spacing. A Ti-Sapphire laser was used to initiate the two-photon polymerization process, and masters were fabricated using a laser write speed of 100 μm/sec and a power of 370 mW (measured at the output of laser). The laser was operated at 800 nm and 76 MHz with a 150 fs pulse duration. Masters were fabricated onto polymer substrates made via a microstereolithography system, as previously described; and both masters and their substrates were made from a commercially available class 2a biocompatible UV curable polymer (E-shell® 300, a) (see, e.g., Miller P R et al., “Integrated carbon fiber electrodes within hollow polymer microneedles for transdermal electrochemical sensing,” Biomicrofluidics 2011 Mar 30;5(1):1341). Fabricated structures were developed in ethanol and post-cured with a UV lamp to ensure complete polymerization. Master structures were then coated with 100 nm of gold using a SEM sputter coater depositing at about 0.1 nm/sec.

Master micromolding: Master structures were micromolded with PDMS by using Sylgard® 184 at a ratio of 10:1 or 20:1 of base:curing agent, in which the base included silicone precursors and the curing agent included a catalyst. Structures were placed under vacuum prior to curing of the PDMS in order to remove air bubbles from the face and the ledge of the microneedle masters. Then, the master structures and PDMS were baked in an oven at 100° F. to cure the PDMS. The cured PDMS molds were then released from the master structures.

Electroforming: PDMS molds were coated with a metal multi-layer of 10 nm titanium and 100 nm gold using an e-beam evaporator to create the initial seed layer. Following seed layer deposition, metal coated PDMS molds were electroplated using pulsed chronoamperometry with a VoltaLab potentiostat against an Ag/AgCl reference and Pt wire counter electrode. Either nickel or iron baths were used to electroform the microneedles. Contents for the nickel bath were 300 g/L nickel sulfamate, 11 g/L nickel chloride, 30 g/L boric acid, and 4 g/L sodium dodecyl sulfate; and the bath was heated to 37° C. while stirring. Contents for the iron bath were 120 g/L iron sulfate, 45 g/L boric acid, and 0.5 g/L ascorbic acid; and the bath was maintained at 25° C. while stirring. Baths were remade for each sample.

Both constant plating and pulse plating methods were explored for the electroforming process. Prior to the deposition of the bulk of the metal film, an initial plating process took place for 15 minutes at −1.0 V. The sample was then spun such that the electrical connector from the potentiostat was connected to the thicker layer of nickel, which created a more robust connection. Constant plating was performed by applying a potential of −1000 mV for between 2 and 8 hours. For pulsed plating, potentials were cycled between −1.0 V for 3 seconds and −0.5 V for 6 seconds for 4000-6000 repetitions. Electroforms were removed from PDMS molds by hand.

Insertion test: Porcine skin was used for microneedle penetration studies, and tissue samples were acquired from a local abattoir. The tissue samples were acquired immediately following animal sacrifice and stored at −20° C. until use. The porcine skin was thawed in 1× phosphate-buffered saline and then shaved. Hollow metal microneedle arrays were attached to a microfluidic chip made with a previously described technique (see, e.g., Miller P R et al., “Microneedle-based transdermal sensor for on-chip potentiometric determination of K(+),” Adv. Healthc. Mater. 2014 Jun;3(6):876-81). Microneedles were applied to the porcine skin with the Medtronic MiniMed Quick-Serter® at a rate of 0.5 m/s (see, e.g., Miller P R et al., “Electrodeposited iron as a biocompatible material for microneedle fabrication,” Electroanalysis 2015 doi: 10.1002/elan.201500199). Following insertion into the skin, Trypan Blue was injected into the tissue by hand application from a syringe and PEEK tube attachment on the microfluidic chip. The tissue samples were cleaned with an ethanol wipe and imaged using a camera.

Mechanical Testing: The fracture forces for deformation of the hollow metal microneedles were tested using the Bose Electroforce® 3100 mechanical testing instrument (Bose Corp., Framingham, Mass.) with a 20 N load cell. Compressions were conducted at 0.1 mm/s; and microneedles were pressed against a metal platen while monitoring the change in force and the displacement upon impact.

Scanning electron microscopy (SEM) and energy-dispersive X-ray spectroscopy (EDX) imaging: Hollow metal microneedles were imaged using a Carl Zeiss Supra™ 55VP SEM at 10 kV and 15 kV and a working distance of 8.5 mm. All samples were coated with a thin layer of platinum prior to imaging. EDX was used to determine the elemental composition of the deposited metals.

Digital imaging: Optical images of hollow microneedles, molds, cross-sections of molds, and master structures were taken with a Keyence Digital Microscope. All other optical images were taken with a digital camera.

### Example 3

### Optimization and Design of Masters

Two-photon polymerization employing laser direct write was created to improve upon previous fabrication systems by allowing the polymerization process to happen within the resin as opposed to at the surface of the resin as seen in traditional UV-based stereolithography systems (see, e.g., Cumpston B H et al., “Two-photon polymerization initiators for three-dimensional optical data storage and microfabrication,” Nature 1999;398(6722):51-4). The multi-photon process lends towards high spatial resolution and can create structures with sub-100 nm resolution (see, e.g., Wollhofen R et al., “120 nm resolution and 55 nm structure size in STED-lithography,” Opt. Express 2013 May 6;21(9):10831-40). This technique has been used to create hollow microneedles with well-defined control of microneedle geometry however scaling this system for fabrication of large arrays of microneedles is troublesome (see, e.g., Doraiswamy A et al., “Two photon induced polymerization of organic-inorganic hybrid biomaterials for microstructured medical devices,” Acta Biomater. 2006;2(3):267-75).

The main issues facing this technique is fabrication time due to the single focal spot, alignment between bores in the substrate and bores of the microneedles, and yield of the successfully created microneedles since one poorly produced structure can ruin an entire array. Components such as multi-spot two-photon polymerization have been incorporated into this fabrication technology to increase its scalability, where multiple spots (4×4 array) can be fabricated at the same time, however alignment between the substrate and microneedles is still an issue for hollow microneedles (see, e.g., Obata K et al., “Multi-focus two-photon polymerization technique based on individually controlled phase modulation,” Opt. Express 2010;18(16):17193-200; and Gittard S D et al., “Fabrication of microscale medical devices by two-photon polymerization with multiple foci via a spatial light modulator,” Biomed. Opt. Express 2011;2(11):3167-78). These factors influenced a new method to be created for fabrication of arrays of hollow microneedles with the particular features previously listed (e.g. off-set bore, sharp tip).

We choose to take advantage of the resolution and feature compatibility of 2PP-LDW to make master structures, which could then be turned into hollow microneedles following a molding step. Our goal was to create a method that was scalable, used biocompatible materials, created microneedles with the desired geometries, and the master structure could be fabricated via other, cheaper fabrication systems.

Kim et al. showed that replication of high aspect ratio structures could be created via electroplating into PDMS molds (see, e.g., Kim K et al., “Rapid replication of polymeric and metallic high aspect ratio microstructures using PDMS and LIGA technology,” Microsys. Technol. 2002;9(1-2):5-10). Molds of high aspect ratio structures fabricated with LIGA were made with PDMS and coated with a seed layer then electroplate for creation of the metallic replica. The technique demonstrated a rapid method for mass replication of structures however the approach showcased only uniform structures without selectively placed voids. Additionally, reusability of the molds following the electroplating step was not investigated.

We sought to extend known technique for making hollow microneedles, and creation of the bore was the first aspect investigated. FIG. shows a schematic of the proposed fabrication process, which we refer to as electromolding, using an inward facing ledge for creation of the microneedle bore. Due to the directional nature of evaporative metal deposition, Kim et al. ensured the side walls of their molds were coated by tilting and rotating the sample during seed layer deposition. We chose to use the directionality of metal evaporation to our advantage and designed features on the microneedle master to either block or catch some portion of the seed layer to create a void in the seed layer for the microneedle bore.

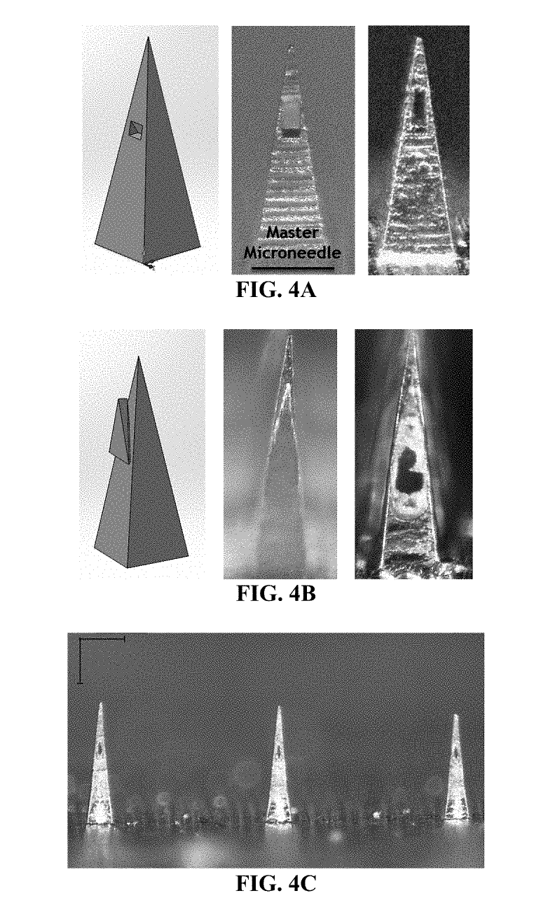

A four sided pyramidal microneedle structure was chosen due to the simply geometry amenable to micromolding and the angled walls would be easily coated with the seed layer deposition. Microneedle master designs using vertical side walls suffered from inconsistent coating. Two different microneedle master structures were designed (FIG. 4A-4C). The first design (FIG. 4A) included an inward facing ledge on one of the pyramidal microneedle faces, designed to block a portion of the seed layer. The second design (FIG. 4B) included an outward facing structure, designed to catch some portion of the seed layer. FIG. 4C shows an array of microneedles.

After fabricating each of the microneedles, it became apparent that the inward facing ledge was a simpler structure to fabricate repeatedly for creating the off-set bore. In addition, the outward facing structure didn't provide sharp enough angles to avoid deposition of the seed layer in the void and was a structure that would be very difficult to replicate with another, cheaper fabrication system. Based on these results, the inward facing ledge design was further studied.

### Example 4

### Effects of Seeding and Electroplating Conditions on Molds Having Ledges

Seed layer material and thickness were investigated. Prior studies employed a thick (2 μm) seed layer of either of Cu or Ti/Au. Due to copper's known cytotoxicity, Ti/Au was chosen for this study. PDMS molds were coated with varying thicknesses; and a seeding layer including 10 nm/100 nm Ti/Au was tested for their ability to remain adherent to the mold, film electrical resistance, and removal from mold post electroplating without tearing off portions of PDMS. Electrical resistance measurements were performed using an Ohm meter across the length of the mold (2 cm) and showed no significant difference (10-20 Ohm) in resistance for each of the seed layer thicknesses tested. The 10 nm/100 nm Ti/Au thickness was chosen to try and reduce the amount of seed layer deposited behind the ledge, thus retaining the largest bore size possible. Each of the seed layer thicknesses created uniform metal deposition on the molds and were capable of being removed without destroying the mold or pulling any noticeable portion of PDMS from the mold. Additionally, electroforms stayed adhered to the molds during the plating process and didn't delaminated due to the plating process.

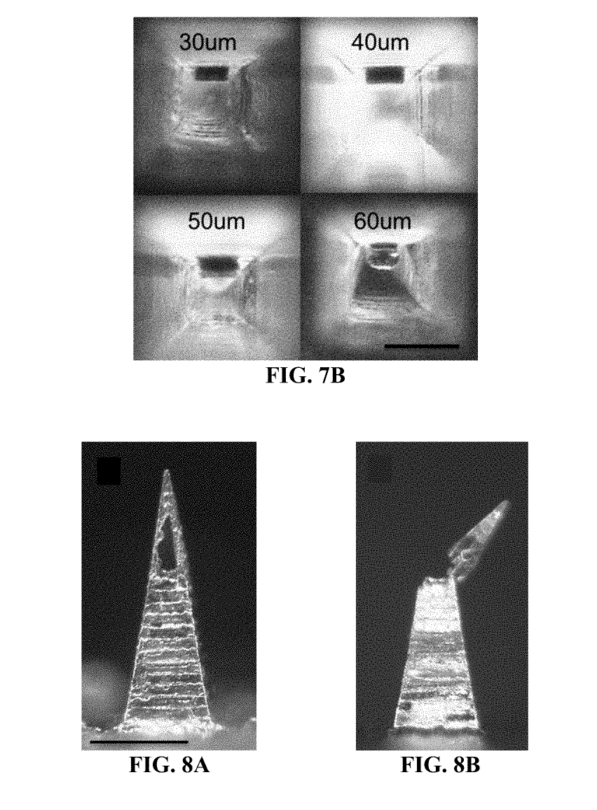

During the initial seed layer thickness experiments, inward facing ledges were used with varying ledge depths and were shown to affect the size of the resulting bore. Ledge depths that resulted in perpendicular faces created smaller bores, as anticipated, compared to deeper ledges. Since these ledges were not angled back, away from the incoming e-beam seed layer deposition most of the ledge was covered with the seed layer and reduced the size of the microneedle bore. Based on these results, we sought to adjust the ledge depth of the inward facing ledge and investigate the resulting seed layer void. Ledge sizes of 30 μm, 40 μm, 50 μm, and 60 μm were incorporated into master microneedle structures and PDMS micromolded. FIG. 7B shows optical images taken from within a single microneedle mold detailing the size of each of the ledges. It was noticed that the size of the microneedle ledges did not exactly reflect the input size of the ledge designed in SolidWorks®, and FIG. 7A shows the relation between input ledge size and resulting mold ledge size. In 2PP-LDW, as is with other rapid prototyping techniques, the input size of the structure doesn't always directly reflect size of the fabricated part. In this case, we believe that the fabrication parameters are typically optimized for fabrication time and that the mechanical shutter can lag behind the path of the beam especially when making small structures as seen with the ledges.

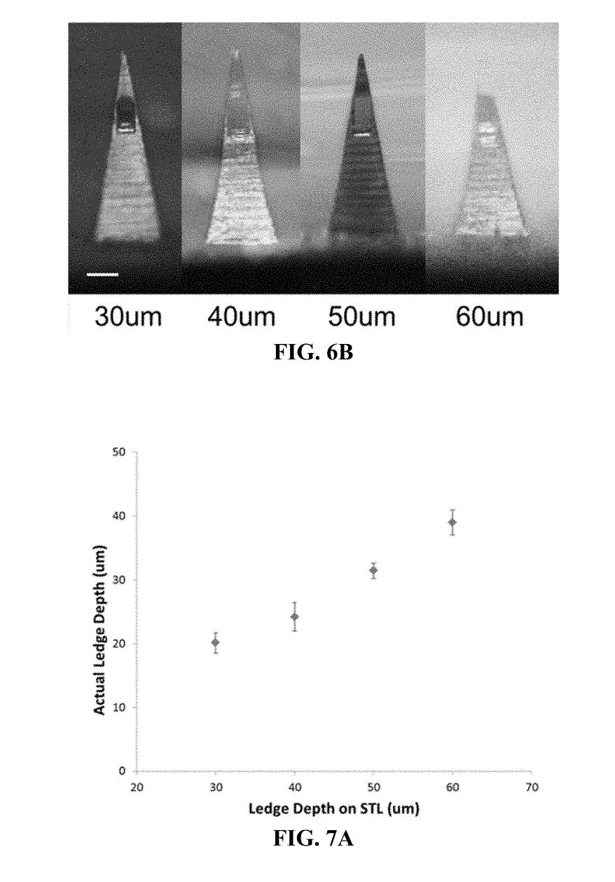

Ledge size effect on resulting seed layer void was investigated as seen in FIG. 6A-6B. PDMS molds were created from microneedle masters having each of the bore sizes (30 μm, 40 μm, 50 μm, and 60 μm) and coated with 10 nm/100 nm Ti/Au. Molds were then cut directly beside the microneedle mold on the ledge side and were imaged to determine the effect of the ledge size on resulting seed layer void. As anticipated, increasing the ledge size created larger voids however the 60 μm ledge had no seed layer deposited on the tip. Upon investigation it was apparent that at this size the microneedle could no longer withstand solvent developing following 2PP-LDW fabrication and caused the microneedle master the bend at the ledge due to the lack of supporting polymer. This ledge size was not investigated following this result. Each ledge size resulted in a consistent void with bore void heights ranging from ˜60 μm to ˜130 μm depending on the ledge size. The 40 μm and 50 μm ledge size were studied moving forward due to the size of their resulting void.

### Example 5

### Improvements to Tip Survivability for Microneedles

It became apparent during fabrication of the hollow microneedles that some of the microneedle tips were not surviving the mold removal process. Upon imaging within the molds following the electroplating step, metal was noticed at the tip of the mold indicating that either a suitable amount of metal was not deposited at that area or something was causing tip to shear from the rest of the microneedle. Two aspects were investigated to improve tip survivability and first the PDMS mold precursor ratio was adjusted to create a more elastic mold. Previous groups have studied the effect of adjusting the ratio of cross-linker to polymer precursor on resulting Young's Modulus and Brown et al. showed adjusting the ratio from 10:1 to 50:1 the modulus was reduced from 1783 kPa to 48 kPa (see, e.g., Brown W Q et al., “Evaluation of polydimethylsiloxane scaffolds with physiologically-relevant elastic moduli: interplay of substrate mechanics and surface chemistry effects on vascular smooth muscle cell response,” Biomaterials 2005;26(16):3123-9).

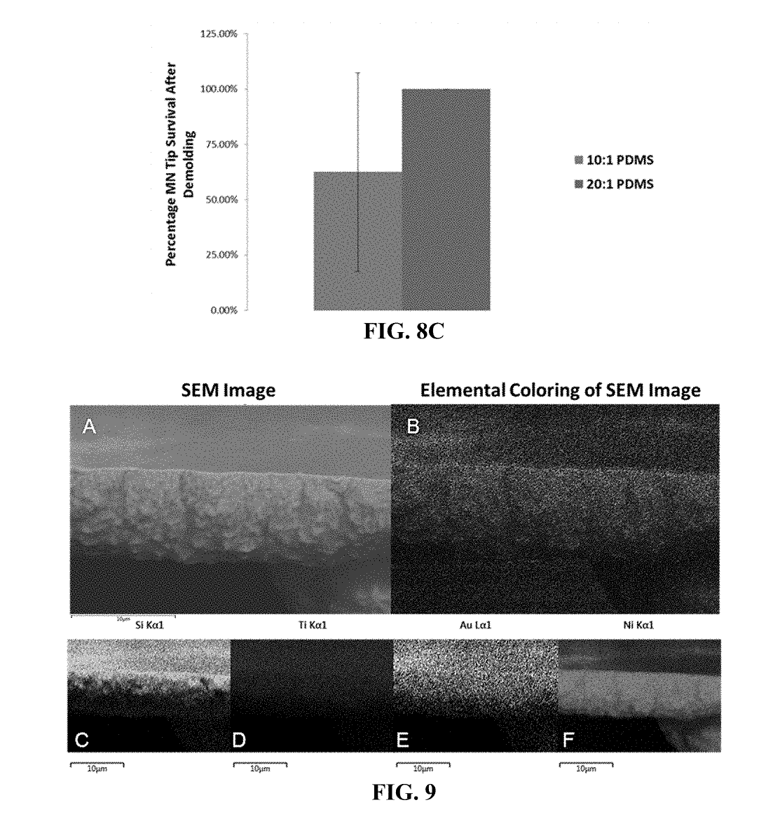

We studied cross-linker to polymer precursor ratios of 10:1, 20:1, 30:1, and 50:1. Molds resulting from the 50:1 PDMS precursor ratio did not survive removal from the laminate molds they were cast in and tore into pieces upon removal. Molds with 30:1 ratio were capable of surviving the casting removal process however were difficult to handle and caused island formation of the seed layer due to their elasticity which made establishing an electrical connection difficult. Compared to the 10:1 PDMS mold ratio the 20:1 mold ratio were elastic however not too deformable to effect the electrical stability of the seed layer. The elastic modulus of these two molds was tested using nanoindentation for the 10:1 and 20:1 molds. Tip survival, meaning intact tip upon mold removal, was compared for each of the molds. The 20:1 mold resulted in no tip deformation when 50 μm ledges were electroplated for 4000 cycles of 3 second pulse of −0.1 V, followed by 6 second pulse of −0.5 V in a heated nickel bath against a Ag/AgCl reference and Pt wire counter electrode as seen in FIG. 8A-8C.

The second method to improve tip survivability was adjusting the electroplating parameters. Initial tests used constant potential plating to form the hollow microneedles. While this technique was effective for forming the microneedles overplating was noticed at the edges of the ledge and mold which in some cases caused closure of the bore from within the mold.

A pulsed electroplating technique was investigated to improve distribution of plating salts across the surface of the microneedle mold since points and edges preferentially plate and result in non-uniform surfaces. Additionally, we believe that due to the depth of the microneedle tips metal salts replenish slower compared to the substrate or portions of the microneedle mold towards the base. Pulsed electroplating techniques cycle the reducing potential or current between values that deposit the metal and one that doesn't not so as to allow the redistribution of ions to take place across the sample (see, e.g., Chandrasekar M S et al., “Pulse and pulse reverse plating—conceptual, advantages and applications,” Electrochim. Acta 2008;53(8):3313-22).

Comparison between constant plating at −1.0V and pulsing between −1.0V and −0.5V (not a potential capable of electroplating either bath) resulted in less overdeposition of hollow microneedles and contributed to stronger tip due to the lack of tip deformation using this technique. Additionally, resulting current densities during pulsed deposition (−25 mA/cm2) were comparable previous reports that showed less residual stress within pulsed electroplated films compared to constant plating conditions at the same current density (see, e.g., Hadian S E et al., “Residual stresses in electrodeposits of nickel and nickel-iron alloys,” Surf Coatings Technol. 1999;122(2-3):118-35).

### Example 6

### Reusability of Molds After Microneedle Formation

As we began creating hollow microneedles using the electromolding method, it was noticed that the molds seemed unaffected by the process and experiments were performed to determine whether they were damaged at all. Upon removal of the a microneedle from the mold, SEM and EDX color mapping was used to determine whether any portion of the PDMS mold was removed and stayed on the microneedle. FIG. 9 shows cross-sectional SEM images and false colored elemental images from the bore of an electromolded hollow microneedle. Cross-sections were imaged to estimate layer thickness of each of the deposited coatings (titanium, gold, and nickel, FIG. 9D-9E) and any residual material (silicon, FIG. 9C) from the molding process.

EDX elemental mapping showed the three distinct metal layers from the seed layer and electroplating process and a very thin layer of silicon on the surface of the Ti layer resulting from contact with the mold. While this imaging technique is not designed to be quantitative the color intensity of the silicon layer was comparable to that of the titanium layer, which was deposited at 10 nm. This result suggests that removal of the microneedles from the mold doesn't significantly destroy the molds and motivated experiments on reusing the molds. Initial experiments performed with previously used molds resulted in hollow microneedles that mimicked the first batch made and further experiments are needed to determine the number of times the molds can be reused before producing unacceptable microneedles.

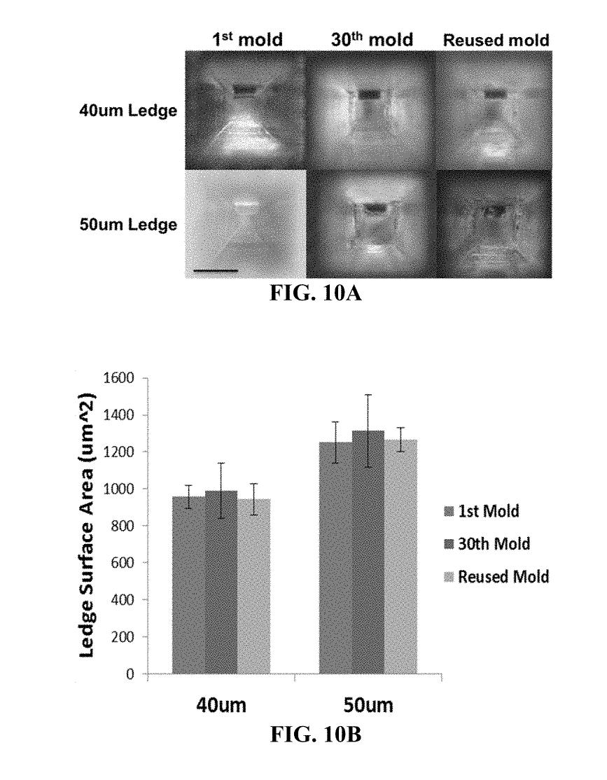

Experiments were performed to determine what effects if any happens to the mold following reuse and after numerous replications of the master microneedle structures. Optical images of the ledge within the mold were taken both after microneedle removal and following replications of the master as seen in FIG. 10A. Ledge integrity was investigated due to its role in creation of the microneedle bore. Once the ledge loses its structure, the mold no longer becomes effective in producing microneedles with the required features. Using the 40 μm and 50 μm ledges, size molds were imaged before and after electromolding; and no damage was noticed to the ledge of the mold following microneedle removal. The 40 μm and 50 μm ledges were monitored over the course of mold replication (FIG. 10B); and 31 molds were created without noticeable feature deterioration indicating a potential high degree of scalability for this technique.

Overall, described herein is an exemplary fabrication method to create flexible hollow microneedle arrays from a reusable master and reusable molds. Two-photon polymerization utilizing laser direct write was used for creation of microneedle master structures. Molds were made from master structures and electroplated into for formation of the hollow microneedles. Features used for creation of the microneedle bore were investigated for the type of ledge structure used, their ability to be molded, and size of resulting seed layer void. Molds were capable of being reused, molded over thirty times without damaging, and not losing significant portions of material when microneedles were removed. Electroformed microneedles were tested their tip fracture strength and for delivery into ex vivo porcine skin. The electroforming method presented showcases a scalable method for creation of flexible hollow microneedle arrays.

### Example 7

### Properties of Electroformed Microneedles and its Substrate

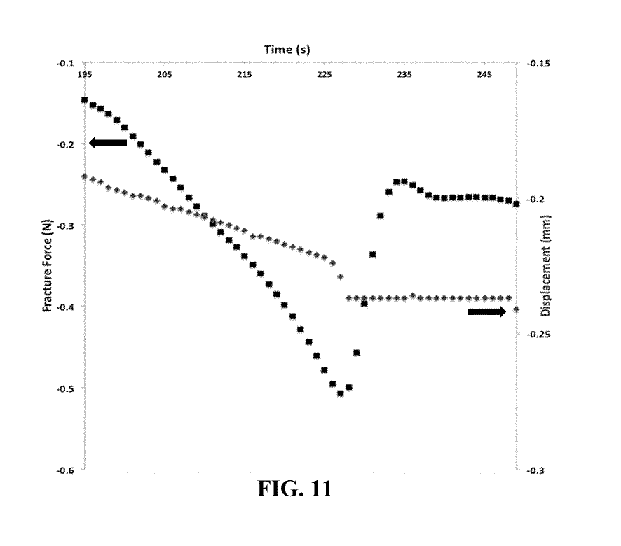

Electromolded hollow microneedles were tested in terms of the mechanical properties and their functionality. Compression tests were performed using the ElectroForce® 3100 system by compressing a microneedle while monitoring both the displacement of the platens and the resulting force. Microneedles were compressed using a previous method and resulted in consistent fracture forces and average fractures for microneedles made with 4000 pulses between −1.0 V for 3 seconds and −0.5 V for 6 seconds (FIG. 11). Functionality testing of the microneedles was performed by insertion and injection experiments with ex vivo porcine skin. Hollow microneedle arrays were attached to a laminate microfluidic chip and were inserted into the porcine skin using a controlled velocity of ˜0.5 m/s by the Medtronic MiniMed® Quick-serter®. Once in the skin, Trypan blue with injected via hand pressure applied to a syringe connected to the microfluidic chip. Porcine skin samples were cleaned with an alcohol swipe and imaged for their insertion sites. A 3×1 microneedle array was applied to the skin with injection sites seen for each microneedle. Upon removal from the skin, microneedles remained intact and were capable of multiple injections prior to tip deformation.

It anticipated that a flexible nearly conformal substrate would benefit hollow microneedle functionality for sensing applications due to the elastic nature of the skin and dynamic movements seen over the course of a day for the end used. Due to these facts electromolded hollow microneedle arrays were tested for their ability to withstand multiple types of deformation that may be seen during the course of use. As seen in FIG. 12A-12C, arrays were bent in multiple directions and twisted. Electroformed microneedle arrays were capable of deforming with the direction of the bend or twist without any fracture or tearing of the microneedles or their substrate. Following these movements the array returned to its original shape. These results indicate that the metal multilayer substrate provides a strong substrate for the microneedle array while offering a degree of flexibility that may be beneficially to use during dynamic movements.

In conclusion, the electromolding fabrication processes herein combined PDMS micromolding with electroforming for creation of hollow microneedles. Electroforming is the process of creation of structures via electroplating, while electroplating refers to depositing a coating on a preexisting structure. Master structures were first designed in SolidWorks® and then fabricated with a two-photon lithography system using laser direct write with commercially available UV cured polymer. Master structures were then molded with PDMS to create the inverse (or negative replica) of the desired microneedle configuration. A seed layer including 10 nm of titanium and then 100 nm of gold was deposited using e-beam evaporation. Metal-coated PDMS molds were then electroplated into the mold (and onto the seed layer) until a suitable amount of metal was deposited. Then, the electroform was removed. An off-set bore was created by designing the master structure with an inward facing ledge that blocks a portion of the seed layer upon deposition leaving a void on the face of the microneedle.

In some instances, electroplating was employed. Carefully electroplating into the mold with pulse plating allowed for more even metal deposition across the surface of the microneedle. Furthermore, overdepositing was minimized, which also reduced the chance of blocking the bore; and such overdepositing was observed in some circumstances with constant plating techniques. Electroplating into the mold allowed for the tip of the microneedle to remain sharp and not suffer from the “Q-tip” effect, which causes microneedle tips to become dull during electroplating due to preferential plating within corners and points because of higher current densities found in these locations.

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

1. A microneedle array comprising a plurality of electromolded needles arranged on a flexible substrate, wherein each of the electromolded needles comprises an outer layer comprising one or more seeding materials and an inner layer comprising one or more electroplating materials, and wherein each of the electromolded needles comprises an internal hollow bore and an orifice disposed at the distal end of the bore.

2. The array of claim 1, wherein the orifice for each of the electromolded needles is disposed off-center from a center axis of the bore.

3. The array of claim 2, wherein each of the electromolded needles further comprises at least one puncturing edge in proximity to the orifice.

4. The array of claim 1, wherein each of the electromolded needles comprises a pyramidal structure.

5. The array of claim 1, wherein the one or more seeding materials are selected from the group consisting of titanium, gold, copper, nickel, tungsten, and alloys or multilayers thereof.

6. The array of claim 1, wherein the one or more electroplating materials are selected from the group consisting of nickel, iron, aluminum, copper, and alloys or multilayers thereof.

7. The array of claim 1, wherein the orifice is a rectangular, circular, or elliptical orifice.

8. An apparatus comprising a microneedle array of claim 1.

9. The apparatus of claim 8, further comprising:
(i) a sensor component comprising the microneedle array and at least one sensing transducer in fluidic communication with at least one hollow needle of the microneedle array, wherein the at least one sensing transducer is configured to detect one or more markers in the sample; and
(ii) an electronic component comprising circuitry configured for signal processing, signal control, power control, and/or communication signaling, wherein the electronic component is connected electrically to the sensor and delivery components.

10. The apparatus of claim 9, wherein (i) the sensor component comprises:
a plurality of hollow needles, wherein each needle has an interior surface facing the hollow lumen and an exterior surface, the distal end of the exterior surface for at least one needle comprises a puncturing edge, and at least one needle has a length of more than about 0.5 mm;
a substrate coupled to the plurality of hollow needles, wherein the substrate comprises one or more inlets in fluidic communication with the proximal end of at least one needle;
a first channel coupled to the substrate and in fluidic communication with at least one inlet of the substrate; and
one or more sensing transducers in fluidic communication with the first channel.

11. The apparatus of claim 8, further comprising:
(iii) a delivery component comprising one or more depots configured to contain one or more therapeutic agents,
wherein the delivery component comprises a plurality of hollow needles, each needle has an interior surface facing the hollow lumen and an exterior surface, the distal end of the exterior surface for at least one needle comprises a puncturing edge, and at least one needle has a length of more than about 0.5 mm;
and wherein at least one needle is in fluidic communication with at least one depot.

12. The apparatus of claim 8, further comprising:
(iv) a fluidic component comprising one or more fluidic channels, chambers, pumps, and/or valves configured to provide fluidic communication between the sensor component and/or the delivery component, if present, and the sample.
